SEQUENCE LISTING (55 sequences)

SEQ ID NO: 1 - DNA, 20 bp, Artificial Sequence
  Synthetic Polynucleotide
gcacagagctgcaaacaact

SEQ ID NO: 2 - DNA, 24 bp, Artificial Sequence
  Synthetic Polynucleotide
aaaagcaaagtcatatacctcacg

SEQ ID NO: 3 - DNA, 20 bp, Artificial Sequence
  Synthetic Polynucleotide
gaaagctcagcagacgacct

SEQ ID NO: 4 - DNA, 20 bp, Artificial Sequence
  Synthetic Polynucleotide
cacaccacggacacacaaag

SEQ ID NO: 5 - DNA, 22 bp, Artificial Sequence
  Synthetic Polynucleotide
gttgtcacacttgtaacaccac

SEQ ID NO: 6 - DNA, 22 bp, Artificial Sequence
  Synthetic Polynucleotide
tgtgcccataagtagttgctgt

SEQ ID NO: 7 - DNA, 20 bp, Artificial Sequence
  Synthetic Polynucleotide
agcaaaaccagacacctcca

SEQ ID NO: 8 - DNA, 20 bp, Artificial Sequence
  Synthetic Polynucleotide
agcaaaaccagacacctcca

SEQ ID NO: 9 - DNA, 20 bp, Artificial Sequence
  Synthetic Polynucleotide
ttgcatttggaacctcagaa

SEQ ID NO: 10 - DNA, 20 bp, Artificial Sequence
  Synthetic Polynucleotide
tgcttcatcgttttcctcct

SEQ ID NO: 11 - DNA, 23 bp, Artificial Sequence
  Synthetic Polynucleotide
gagcacacaagtagatattcgca

SEQ ID NO: 12 - DNA, 20 bp, Artificial Sequence
  Synthetic Polynucleotide
agtagaacagttggggcaca

SEQ ID NO: 13 - DNA, 24 bp, Artificial Sequence
  Synthetic Polynucleotide
aggcatgtggatttaaacaaaagg

SEQ ID NO: 14 - DNA, 20 bp, Artificial Sequence
  Synthetic Polynucleotide
ttgtgtttgtctacgtcggg

SEQ ID NO: 15 - DNA, 22 bp, Artificial Sequence
  Synthetic Polynucleotide
caactgacctacactgctatga

SEQ ID NO: 16 - DNA, 20 bp, Artificial Sequence
  Synthetic Polynucleotide
gcttgttctgcttgtccatc

SEQ ID NO: 17 - DNA, 22 bp, Artificial Sequence
  Synthetic Polynucleotide
gtagtagaaacctcgcaagacg

SEQ ID NO: 18 - DNA, 21 bp, Artificial Sequence
  Synthetic Polynucleotide
gcacacaaaggacacacaaag

SEQ ID NO: 19 - DNA, 19 bp, Artificial Sequence
  Synthetic Polynucleotide
aggtgcatgtgttgtctga

SEQ ID NO: 20 - DNA, 21 bp, Artificial Sequence
  Synthetic Polynucleotide
cctgtatagcttcctgctatt

SEQ ID NO: 21 - DNA, 20 bp, Artificial Sequence
  Synthetic Polynucleotide
tatgcgtgaccagctaccag

SEQ ID NO: 22 - DNA, 20 bp, Artificial Sequence
  Synthetic Polynucleotide
aacacctgcaacacggagct

SEQ ID NO: 23 - DNA, 20 bp, Artificial Sequence
  Synthetic Polynucleotide
acagctcagaggatgaggat

SEQ ID NO: 24 - DNA, 21 bp, Artificial Sequence
  Synthetic Polynucleotide
tagcttgtctagcttgctgtg

SEQ ID NO: 25 - DNA, 22 bp, Artificial Sequence
  Synthetic Polynucleotide
cttgtatgtcacgagcaattag

SEQ ID NO: 26 - DNA, 19 bp, Artificial Sequence
  Synthetic Polynucleotide
tgtgacgctgttgttcgtc

SEQ ID NO: 27 - DNA, 21 bp, Artificial Sequence
  Synthetic Polynucleotide
gataatttcacactaaccgcc

SEQ ID NO: 28 - DNA, 20 bp, Artificial Sequence
  Synthetic Polynucleotide
agatgctctgacttgatggt

SEQ ID NO: 29 - DNA, 27 bp, Artificial Sequence
  Synthetic Polynucleotide
tgtgtactgcaagcaacagttactgcg

SEQ ID NO: 30 - DNA, 24 bp, Artificial Sequence
  Synthetic Polynucleotide
cgagcattccagcagctgtttctg

SEQ ID NO: 31 - DNA, 29 bp, Artificial Sequence
  Synthetic Polynucleotide
tgtcaacagtacagcaagtgacctacgaa

SEQ ID NO: 32 - DNA, 25 bp, Artificial Sequence
  Synthetic Polynucleotide
acgtcctgttgtaaatgtgaggcga

SEQ ID NO: 33 - DNA, 26 bp, Artificial Sequence
  Synthetic Polynucleotide
cctgttgacctgttgtgttacgagca

SEQ ID NO: 34 - DNA, 27 bp, Artificial Sequence
  Synthetic Polynucleotide
agctgttaatgggctcatttggaatcg

SEQ ID NO: 35 - DNA, 24 bp, Artificial Sequence
  Synthetic Polynucleotide
atatttcgggtcgttggacagggc

SEQ ID NO: 36 - DNA, 24 bp, Artificial Sequence
  Synthetic Polynucleotide
aggtgacagctcagatgaggagga

SEQ ID NO: 37 - DNA, 24 bp, Artificial Sequence
  Synthetic Polynucleotide
tgcgagccttacagcagctgttta

SEQ ID NO: 38 - DNA, 21 bp, Artificial Sequence
  Synthetic Polynucleotide
aaccgctgtgtccagcagaaa

SEQ ID NO: 39 - DNA, 20 bp, Artificial Sequence
  Synthetic Polynucleotide
cgtagcctgtccagcccgtc

SEQ ID NO: 40 - DNA, 25 bp, Artificial Sequence
  Synthetic Polynucleotide
tgaagtagaccatttgcaggagcgg

SEQ ID NO: 41 - DNA, 25 bp, Artificial Sequence
  Synthetic Polynucleotide
catgcagttaatcaccaccaacatc

SEQ ID NO: 42 - DNA, 17 bp, Artificial Sequence
  Synthetic Polynucleotide
tcttccctcgaacctgc

SEQ ID NO: 43 - DNA, 143 bp, Artificial Sequence
  Synthetic Polynucleotide
aaagttaccacagttatgcacagagctgcaaacaactatacatgatataatattagaatgtgtgtactgcaagcaacagttactgcgacgtgaggtatatgactttgcttttcgggatttatgcatagtatatagagatggga

SEQ ID NO: 44 - DNA, 152 bp, Artificial Sequence
  Synthetic Polynucleotide
tgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggt

SEQ ID NO: 45 - DNA, 141 bp, Artificial Sequence
  Synthetic Polynucleotide
tctacacttcgtttgtgtgtacagagcacacaagtagatattcgcatattgcaagagctgttaatgggctcatttggaatcgtgtgccccaactgttctactagactgtaactacaatggctgatccagcaggtacagatg

SEQ ID NO: 46 - DNA, 143 bp, Artificial Sequence
  Synthetic Polynucleotide
acaaccagccacagctgattactacattgtaacctgttgtcacacttgtaacaccacagttcgtttatgtgtcaacagtacagcaagtgacctacgaaccatacagcaactacttatgggcacagtgaatattgtgtgcccta

SEQ ID NO: 47 - DNA, 153 bp, Artificial Sequence
  Synthetic Polynucleotide
attgacggtccagctggacaagcaaaaccagacacctccaattataatattgtaacgtcctgttgtaaatgtgaggcgacactacgtctgtgtgtacagagcacacacattgacatacgtaaattggaagatttattaatgggcacatttgga

SEQ ID NO: 48 - DNA, 146 bp, Artificial Sequence
  Synthetic Polynucleotide
taatacaaagttatataatttattaataaggtgcatgtgttgtctgaaaccgctgtgtccagcagaaaaattaagacacctaaatagcaaacgaagatttcataaaatagcaggaagctatacaggacagtgtcgacggtgctgga

SEQ ID NO: 49 - DNA, 152 bp, Artificial Sequence
  Synthetic Polynucleotide
aacactgcaagaaattgtattgcatttggaacctcagaatgaattagatcctgttgacctgttgtgttacgagcaattaagcgagtcagaggaggaaaacgatgaagcagatggcgttagtcatgcacaactaccagcccgacgagccgaac

SEQ ID NO: 50 - DNA, 141 bp, Artificial Sequence
  Synthetic Polynucleotide
atttgacagctcagaggaggaggatgaagtagataatatgcgtgaccagctaccagaaagacgggctggacaggctacgtgttacagaattgaagctccgtgttgcaggtgttcaagtgtagtacaactggcagtggaaag

SEQ ID NO: 51 - DNA, 144 bp, Artificial Sequence
  Synthetic Polynucleotide
tatatattagatctgcaacctgaaacaactgacctacactgctatgagcaattaggtgacagctcagatgaggaggatacagatggtgtggaccggccagatggacaagcagaacaagccacaagcaattactacattgtgaca

SEQ ID NO: 52 - DNA, 152 bp, Artificial Sequence
  Synthetic Polynucleotide
cattgtgtccacaagaaaaaaaaaggcatgtggatttaaacaaaaggtttcataatatttcgggtcgttggacagggcgctgtgcagtgtgttggagaccccgacgtagacaaacacaagtgtaacctgtaacaacgccatgagaggaaaca

SEQ ID NO: 53 - DNA, 140 bp, Artificial Sequence
  Synthetic Polynucleotide
gtgtgttgtaagtgtaataatcaacttcagctagtagtagaaacctcgcaagacggattgcgagccttacagcagctgtttatggacacactatcctttgtgtgtcctttgtgtgcagcaaaccagtaacctgcaatggc

SEQ ID NO: 54 - DNA, 147 bp, Artificial Sequence
  Synthetic Polynucleotide
tgagcaattggacagctcagaggatgaggatgaggatgaagtagaccatttgcaggagcggccacagcaagctagacaagctaaacaacatacgtgttacctaatacacgtaccttgttgtgagtgtaagtttgtggtgcagttgga

SEQ ID NO: 55 - DNA, 180 bp, Artificial Sequence
  Synthetic Polynucleotide
gaccttgtatgtcacgagcaattaggagattcagacgatgaaatagatgaacccgaccatgcagttaaccaccaccaacatcaactactagccagacgggacgaacaacagcgtcacacaattcagtgtacgtgttgtaagtgtaacaacctactgcaactagtagtagaagcgtcgcgg

(19) United States
(12) Patent Application Publication
(10) Pub. No.: US 2024/0254573 A1
(43) Pub. Date: Aug. 1, 2024

(54) METHODS AND SYSTEMS FOR HPV DETECTION AND QUANTIFICATION

(71) Applicant: BOARD OF REGENTS, THE UNIVERSITY OF TEXAS SYSTEM, Austin, TX (US)

(72) Inventors: Maura GILLISON, Houston, TX (US); Weihong XIAO, Houston, TX (US)

(73) Assignee: BOARD OF REGENTS, THE UNIVERSITY OF TEXAS SYSTEM, Austin, TX (US)

(21) Appl. No.: 18/554,389

(22) PCT Filed: Apr. 7, 2022
(86) PCT No.: PCT/US2022/023911

§ 371 Date: Oct. 6, 2023

Related U.S. Application Data
(60) Provisional application No. 63/172,400, filed on Apr. 8, 2021.

(51) Int. Cl. C12Q 1/70; C12Q 1/6886

(52) U.S. Cl. CPC C12Q 1/708; C12Q 1/6886

(57) ABSTRACT

Disclosed are methods and systems for detection and quantification of human papilloma virus (HPV) DNA. Aspects of the disclosure are directed to detection of HPV DNA from cell-free biological samples (e.g., serum, plasma). Disclosed are methods for detecting and quantifying HPV DNA from one or more high-risk types including HPV16, HPV18, HPV31, HPV33, HPV35, HPV39, HPV45, HPV51, HPV52, HPV58, HPV59, HPV56, and HPV68. The present disclosure includes compositions (e.g., primers, probes) useful for amplification and detection of HPV DNA.

### CROSS REFERENCE TO RELATED APPLICATIONS

This application claims benefit of priority to U.S. Provisional Application No. 63/172,400, filed Apr. 8, 2021, which is hereby incorporated by reference in its entirety.

### SEQUENCE LISTING

The instant application contains a Sequence Listing which has been submitted in ASCII format and is hereby incorporated by reference in its entirety. Said ASCII copy, created on Apr. 8, 2021, is named MDAC_P1260US_P1_Sequence_Listing.txt and is 13,117 bytes in size.

### BACKGROUND

### I. Field of the Invention

Aspects of this invention relate generally to the fields of cancer biology, virology, and medicine, and specifically relate to methods and systems for detection and analysis of human papilloma virus (HPV), including detection from cell-free samples.

### II. Background

HPV causes 5% of the world-wide tumor burden, accounting for at least 650,000 cases per year. These include all cervical and anal cancers and subsets of other anogenital cancers and head and neck cancers.

Persistent HPV infection is the single most important determinant of risk for development of an HPV-positive cancer, and HPV DNA is always present in HPV-positive cancers. Therefore, HPV DNA detection can be used to identify individuals at risk, diagnose HPV-positive cancers, monitor treatment response, detect minimal residual disease, and to surveil for disease progression. Recently, HPV type-specification has gained increasing importance as several immune therapeutics are targeted to specific HPV types. HPV detection can be performed in rinses or cytology samples collected from the anatomic site from which the cancer arises, in tumor biopsy or resection specimens, and in body fluids including serum and plasma.

Limitations to currently available HPV detection assays include validation limited to specific specimen collection kits and anatomic sites, lack of type-specification, and lack of accurate and precise quantification of viral load. Therefore, none can be used to serially monitor treatment response.

There is a need for methods and systems for detection and quantification of HPV DNA for accurate diagnosis and monitoring of HPV-positive cancers, particularly for HPV DNA from various high-risk types such as HPV16, HPV18, HPV31, HPV33, HPV35, HPV39, HPV45, HPV51, HPV52, HPV58, HPV59, HPV56, and HPV68.

### SUMMARY

Aspects of this disclosure address the needs of patients, clinicians, and researchers by providing methods and compositions for detection and quantification of HPV DNA from various high-risk HPV types, including HPV31, HPV33, HPV35, HPV39, HPV45, HPV51, HPV52, HPV58, HPV59, HPV56, and HPV68. Embodiments of the disclosure are directed to methods for detecting which HPV types from a set of high-risk HPV types are present in a biological sample from a subject, and for quantifying an amount of each HPV subtype in the sample, using at least six (e.g., 6, 7, 8, 9, 10, 11, 12, or 13) primer pairs and/or probes each configured to amplify and detect DNA from a different HPV type. Embodiments include PCR-based methods (e.g., ddPCR, qPCR) for detection and quantification of HPV DNA from a set of high-risk HPV types. Also disclosed are compositions and kits comprising primers and/or probes suitable for amplification of DNA from six or more (e.g., 6, 7, 8, 9, 10, 11, 12, or 13) different high-risk HPV types. For example, in some embodiments, disclosed is a method for detecting which HPV types from a set of 13 high-risk HPV types (HPV16, HPV18, HPV31, HPV33, HPV35, HPV39, HPV45, HPV51, HPV52, HPV58, HPV59, HPV56, and HPV68) are present in a biological sample from a subject using seven primer pairs, each configured to amplify and detect DNA from a different HPV type. The present disclosure also comprises methods for diagnosis and monitoring of HPV-associated cancers using cell-free biological samples (e.g., blood, serum, plasma, etc.).

Embodiments of the present disclosure include methods for detecting HPV DNA, methods for amplifying HPV DNA, methods for quantifying HPV DNA, methods for obtaining amplified HPV DNA, methods for diagnosing HPV-associated cancer, methods for monitoring treatment of HPV-associated cancer, methods for evaluating treatment of HPV-associated cancer, oligonucleotides, as well as polypeptides, primers, probes, kits, and methods of use thereof. Methods of the disclosure can include at least 1, 2, 3, 4, or more of the following steps: obtaining a biological sample, purifying DNA from a biological sample, isolating HPV DNA from a biological sample, partitioning HPV DNA, amplifying HPV DNA from a biological sample, detecting which HPV types are present in a sample, amplifying genomic DNA, generating a signal, detecting a signal, quantifying an amount of HPV DNA in a sample, isolating amplified HPV DNA, and treating a subject for an HPV-associated cancer. Any one or more of the preceeding steps may be excluded from certain embodiments of the disclosure. Kits of the disclosure can include at least 1, 2, 3, or more of the following components: primers, probes, oligonucleotides, amplification reagents, and detection reagents. Any one or more of the preceeding components may be excluded from certain embodiments of the disclosure.

Disclosed herein, in some embodiments, is a method for detecting HPV DNA in a biological sample from a subject, the method comprising (a) amplifying HPV DNA from the biological sample using at least six primer pairs each configured to amplify a different HPV type of a set of high-risk HPV types; and (b) detecting which HPV types of the set of high-risk HPV types are present in the biological sample; wherein the set of high-risk HPV types comprises six or more of HPV16, HPV18, HPV31, HPV33, HPV35, HPV39, HPV45, HPV51, HPV52, HPV58, HPV59, HPV56, and HPV68. In some embodiments, (a) comprises using at least or exactly seven primer pairs, each configured to amplify a different HPV type of a set of high-risk HPV types, and the set of high-risk HPV types comprises HPV16, HPV18, HPV31, HPV33, HPV35, HPV39, HPV45, HPV51, HPV52, HPV58, HPV59, HPV56, and HPV68. Also disclosed herein, in some embodiments, is a method for detecting HPV DNA in a biological sample from a subject, the method comprising (a) amplifying HPV DNA from the biological sample using at least six primer pairs comprising at least six of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26; and (b) detecting the HPV DNA in the biological sample using at least six probes comprising at least six of SEQ ID NOs:29-41.

Further disclosed herein, in some embodiments, is a method for preparing amplified HPV DNA from a biological sample from a subject, the method comprising amplifying HPV DNA from the biological sample using at least six primer pairs comprising at least six of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs: 13 and 14, (viii) SEQ ID NOs: 15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26. In some embodiments, the at least six primer pairs comprises exactly 7, 8, 9, 10, 11, 12, or 13 of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs: 13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26, including any combination thereof. In some embodiments, the at least six probes comprises exactly 7, 8, 9, 10, 11, 12, or 13 of SEQ ID NOs:29-41, including any combination thereof.

In some embodiments, the at least six primer pairs are each configured to amplify a region of the HPV E6 gene or the HPV E7 gene. In some embodiments, (b) comprises using at least six probes each configured to bind to a different HPV type of the set of high-risk HPV types. The set of high-risk HPV types may comprise any combination of 6, 7, 8, 9, 10, 11, 12, or all of HPV16, HPV18, HPV31, HPV33, HPV35, HPV39, HPV45, HPV51, HPV52, HPV58, HPV59, HPV56, and HPV68. Any one or more of the preceeding HPV types may be excluded from embodiments of the disclosure.

In certain embodiments, methods of the disclosure comprise amplifying HPV DNA from a biological sample using at least, at most, or exactly 6, 7, 8, 9, 10, 11, 12, or 13 primer pairs, each configured to amplify a different HPV type of a set of high-risk HPV types. In some embodiments, methods of the disclosure comprise detecting HPV DNA from a biological sample using at least, at most, or exactly 6, 7, 8, 9, 10, 11, 12, or 13 probes, each configured to bind to a different HPV type of a set of high-risk HPV types.

In some embodiments, the set of high-risk HPV types comprises seven or more of HPV16, HPV18, HPV31, HPV33, HPV35, HPV39, HPV45, HPV51, HPV52, HPV58, HPV59, HPV56, and HPV68. In some embodiments, the set of high-risk HPV types comprises eight or more of HPV16, HPV18, HPV31, HPV33, HPV35, HPV39, HPV45, HPV51, HPV52, HPV58, HPV59, HPV56, and HPV68. In some embodiments, the set of high-risk HPV types comprises nine or more of HPV16, HPV18, HPV31, HPV33, HPV35, HPV39, HPV45, HPV51, HPV52, HPV58, HPV59, HPV56, and HPV68. In some embodiments, the set of high-risk HPV types comprises ten or more of HPV16, HPV18, HPV31, HPV33, HPV35, HPV39, HPV45, HPV51, HPV52, HPV58, HPV59, HPV56, and HPV68. In some embodiments, the set of high-risk HPV types comprises eleven or more of HPV16, HPV18, HPV31, HPV33, HPV35, HPV39, HPV45, HPV51, HPV52, HPV58, HPV59, HPV56, and HPV68. In some embodiments, the set of high-risk HPV types comprises twelve or more of HPV16, HPV18, HPV31, HPV33, HPV35, HPV39, HPV45, HPV51, HPV52, HPV58, HPV59, HPV56, and HPV68. In some embodiments, the set of high-risk HPV types comprises HPV16, HPV18, HPV31, HPV33, HPV35, HPV39, HPV45, HPV51, HPV52, HPV58, HPV59, HPV56, and HPV68. In some embodiments, the at least six primer pairs comprise at least 6, 7, 8, 9, 10, 11, or 12, or all, of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26. Any one or more of the preceeding primer pairs may be excluded from certain embodiments of the disclosure.

In some embodiments, the at least six primer pairs comprise at least six of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs: 13 and 14, (viii) SEQ ID NOs: 15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26. In some embodiments, the at least six primer pairs comprise at least seven primer pairs comprising at least seven of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs: 13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26. In some embodiments, the at least six primer pairs comprise at least eight primer pairs comprising at least eight of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26. In some embodiments, the at least six primer pairs comprise at least nine primer pairs comprising at least nine of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs: 15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26. In some embodiments, the at least six primer pairs comprise at least ten primer pairs comprising at least ten of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs: 15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26. In some embodiments, the at least six primer pairs comprise at least eleven primer pairs comprising at least eleven of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs: 13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26. In some embodiments, the at least six primer pairs comprise at least twelve primer pairs comprising at least twelve of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs: 17 and 18, (x) SEQ ID NOs: 19 and 20, and (xi) SEQ ID NOs:21 and 22, (xii) SEQ ID NOs:23 and 25, and (xiii) SEQ ID NOs:24 and 26. In some embodiments, the at least six primer pairs comprise at least thirteen primer pairs comprising (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs: 13 and 14, (viii) SEQ ID NOs: 15 and 16, (viv) SEQ ID NOs:17 and 18, (x) SEQ ID NOs:19 and 20, and (xi) SEQ ID NOs:21 and 22, (xii) SEQ ID NOs:23 and 25, and (xiii) SEQ ID NOs:24 and 26.

In some embodiments, a first primer pair of the at least six primer pairs is configured to amplify HPV16 DNA. In some embodiments, the detecting of (b) comprises using a first probe comprising SEQ ID NO:29. In some embodiments, the first primer pair comprises a first forward primer comprising SEQ ID NO: 1 and a first reverse primer comprising SEQ ID NO:2. In some embodiments, a second primer pair of the at least six primer pairs is configured to amplify HPV18 DNA. In some embodiments, the detecting of (b) comprises using a second probe comprising SEQ ID NO:30. In some embodiments, the second primer pair comprises a second forward primer comprising SEQ ID NO:3 and a second reverse primer comprising SEQ ID NO:4. In some embodiments, a third primer pair of the at least six primer pairs is configured to amplify HPV33 DNA. In some embodiments, the detecting of (b) comprises using a third probe comprising SEQ ID NO:31. In some embodiments, the third primer pair comprises a third forward primer comprising SEQ ID NO:5 and a third reverse primer comprising SEQ ID NO:6. In some embodiments, a fourth primer pair of the at least six primer pairs is configured to amplify HPV35 DNA. In some embodiments, the detecting of (b) comprises using a fourth probe comprising SEQ ID NO:32. In some embodiments, the fourth primer pair comprises a fourth forward primer comprising SEQ ID NO:7 and a fourth reverse primer comprising SEQ ID NO:8. In some embodiments, a fifth primer pair of the at least six primer pairs is configured to amplify HPV45 DNA. In some embodiments, the detecting of (b) comprises using a fifth probe comprising SEQ ID NO:33. In some embodiments, the fifth primer pair comprises a fifth forward primer comprising SEQ ID NO:9 and a fifth reverse primer comprising SEQ ID NO:10. In some embodiments, a sixth primer pair of the at least six primer pairs is configured to amplify HPV31 DNA. In some embodiments, the detecting of (b) comprises using a sixth probe comprising SEQ ID NO:34. In some embodiments, the sixth primer pair comprises a sixth forward primer comprising SEQ ID NO:11 and a sixth reverse primer comprising SEQ ID NO:12. In some embodiments, the at least six primer pairs comprise at least seven primer pairs. In some embodiments, a seventh primer pair of the at least seven primer pairs is configured to amplify HPV58 DNA. In some embodiments, the detecting of (b) comprises using a seventh probe comprising SEQ ID NO:35. In some embodiments, the seventh primer pair comprises a seventh forward primer comprising SEQ ID NO:13 and a seventh reverse primer comprising SEQ ID NO:14. In some embodiments, the at least six primer pairs comprise at least eight primer pairs. In some embodiments, an eighth primer pair of the at least eight primer pairs is configured to amplify HPV52 DNA. In some embodiments, the detecting of (b) comprises using an eighth probe comprising SEQ ID NO:36. In some embodiments, the eighth primer pair comprises an eighth forward primer comprising SEQ ID NO:15 and an eighth reverse primer comprising SEQ ID NO: 16. In some embodiments, the at least six primer pairs are at least nine primer pairs. In some embodiments, a ninth primer pair of the at least nine primer pairs is configured to amplify HPV59 DNA. In some embodiments, the detecting of (b) comprises using a ninth probe comprising SEQ ID NO:37. In some embodiments, the ninth primer pair comprises a ninth forward primer comprising SEQ ID NO: 17 and a ninth reverse primer comprising SEQ ID NO:18. In some embodiments, the at least six primer pairs are at least ten primer pairs. In some embodiments, a tenth primer pair of the at least ten primer pairs is configured to amplify HPV39 DNA. In some embodiments, the detecting of (b) comprises using a tenth probe comprising SEQ ID NO:38. In some embodiments, the tenth primer pair comprises a tenth forward primer comprising SEQ ID NO:19 and a tenth reverse primer comprising SEQ ID NO:20. In some embodiments, the at least six primer pairs are at least eleven primer pairs. In some embodiments, an eleventh primer pair of the at least eleven primer pairs is configured to amplify HPV51 DNA. In some embodiments, the detecting of (b) comprises using an eleventh probe comprising SEQ ID NO:39. In some embodiments, the eleventh primer pair comprises an eleventh forward primer comprising SEQ ID NO:21 and an eleventh reverse primer comprising SEQ ID NO:22. In some embodiments, the at least six primer pairs are at least twelve primer pairs. In some embodiments, a twelfth primer pair of the at least twelve primer pairs is configured to amplify HPV56 DNA. In some embodiments, the detecting of (b) comprises using a twelfth probe comprising SEQ ID NO:40. In some embodiments, the twelfth primer pair comprises a twelfth forward primer comprising SEQ ID NO:23 and a twelfth reverse primer comprising SEQ ID NO:25. In some embodiments, the at least six primer pairs are at least thirteen primer pairs. In some embodiments, a thirteenth primer pair of the at least thirteen primer pairs is configured to amplify HPV68 DNA. In some embodiments, the detecting of (b) comprises using a thirteenth probe comprising SEQ ID NO:41. In some embodiments, the thirteenth primer pair comprises a thirteenth forward primer comprising SEQ ID NO:23 and a thirteenth reverse primer comprising SEQ ID NO:25. In some embodiments, the method further comprises amplifying genomic DNA from the biological sample using an additional primer pair, wherein the additional primer pair is configured to amplify a region of Endogenous Retrovirus Group 3 (ERV-3). In some embodiments, the additional primer pair amplifies a region of ERV-3 comprising SEQ ID:LLL. In some embodiments, the detecting of (b) comprises using an additional probe comprising SEQ ID NO:42. In some embodiments, the additional primer pair comprises an additional forward primer comprising SEQ ID NO:27 and an additional reverse primer comprising SEQ ID NO:28. In some embodiments, the detecting of (b) comprises using 6, 7, 8, 9, 10, 11, 12, or all, of the probes comprising SEQ ID NOs:29-41.

In some embodiments, the biological sample is a blood, plasma, or serum sample. In some embodiments, the HPV DNA is from one or more tumor cells. In some embodiments, the HPV DNA is cell-free DNA. In some embodiments, the biological sample is a tissue sample. In some embodiments, the biological sample is a saliva sample. In some embodiments, the biological sample contains less than 100, 90, 80, 70, 60, 80, 60, 50, 40, 30, 29, 28, 27, 26, 25, 24, 23, 22, 21, 20, 19, 18, 17, 16, 15, 14, 13, 12, 11, 10, 9, 8, 7, or 6 copies of HPV DNA. In some embodiments, the biological sample contains less than 100 copies of HPV DNA. In some embodiments, the biological sample contains less than 20 copies of HPV DNA. In some embodiments, the biological sample contains less than 10 copies of HPV DNA. In some embodiments, the biological sample contains less than 6 copies of HPV DNA. In some embodiments, the method further comprises, prior to amplifying the HPV DNA, partitioning the HPV DNA into a plurality of partitionsIse the plurality of partitions is a plurality of droplets. In some embodiments, (b) comprises detecting one or more signals from the plurality of partitions. In some embodiments, the method further comprises quantifying an amount of each of the HPV types in the biological sample.

In some embodiments, the method further comprises diagnosing the subject with an HPV-associated cancer. In some embodiments, the HPV-associated cancer is head and neck squamous cell carcinoma. In some embodiments, the method further comprises treating the subject for the HPV-associated cancer. Treating a subject for an HPV-associated cancer may comprise, for example, chemotherapy, radiotherapy, surgery, immunotherapy, or a combination thereof. Various methods for treatment of HPV-associated cancers are known in the art and are contemplated herein.

Further disclosed herein, in some embodiments, is a kit for detecting HPV DNA from a subject comprising at least six primer pairs comprising at least six of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs: 15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26. In some embodiments, the at least six primer pairs comprise at least seven primer pairs comprising at least seven of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs: 15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26. In some embodiments, the at least six primer pairs comprise at least eight primer pairs comprising at least eight of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26. In some embodiments, the at least six primer pairs comprise at least nine primer pairs comprising at least nine of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs: 13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26. In some embodiments, the at least six primer pairs comprise at least ten primer pairs comprising at least ten of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs: 13 and 14, (viii) SEQ ID NOs: 15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26. In some embodiments, the at least six primer pairs comprise at least eleven primer pairs comprising at least eleven of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs: 15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26. In some embodiments, the at least six primer pairs comprise at least twelve primer pairs comprising at least twelve of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs: 15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26. In some embodiments, the at least six primer pairs comprise at least thirteen primer pairs comprising (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26. Any one or more of the preceeding primer pairs may be excluded from certain embodiments of the disclosure. In some embodiments, the kit further comprises an additional primer pair comprising SEQ ID NOs:27 and 28. In some embodiments, the kit further comprises at least six probes comprising at least six of SEQ ID NOs:29-41. In some embodiments, the at least six probes comprise at least seven probes comprising at least seven of SEQ ID NOs:29-41. In some embodiments, the at least six probes comprise at least eight probes comprising at least eight of SEQ ID NOs:29-41. In some embodiments, the at least six probes comprise at least nine probes comprising at least nine of SEQ ID NOs:29-41. In some embodiments, the at least six probes comprise at least ten probes comprising at least ten of SEQ ID NOs:29-41. In some embodiments, the at least six probes comprise at least eleven probes comprising at least eleven of SEQ ID NOs:29-41. In some embodiments, the at least six probes comprise at least twelve probes comprising at least twelve of SEQ ID NOs:29-41. In some embodiments, the at least six probes comprise at least thirteen probes comprising SEQ ID NOs:29-41. Any one or more of the probes comprising SEQ ID NOs:29-41 may be excluded from certain embodiments of the disclosure.

Throughout this application, the term “about” is used to indicate that a value includes the inherent variation of error for the measurement or quantitation method.

The use of the word “a” or “an” when used in conjunction with the term “comprising” may mean “one.” but it is also consistent with the meaning of “one or more,” “at least one.” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise.” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

Use of the one or more sequences or compositions may be employed based on any of the methods described herein. Other embodiments are discussed throughout this application. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Examples section are understood to be embodiments that are applicable to all aspects of the technology described herein.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

A variety of embodiments are discussed throughout this application. Any embodiment discussed with respect to one aspect applies to other aspects as well and vice versa. Each embodiment described herein is understood to be embodiments that are applicable to all aspects. It is contemplated that any embodiment discussed herein can be implemented with respect to any method or composition, and vice versa. Furthermore, compositions and kits can be used to achieve methods disclosed herein.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows analysis of various annealing/extension temperatures for amplification of DNA using HPV16 and ERV3 primers and probes.

FIG. 2 shows detection of a serial dilution of HPV16 DNA.

FIG. 3 shows quantification of a serial dilution of HPV16 DNA.

FIG. 4 shows detection of a serial dilution of HPV16 DNA in human plasma.

FIG. 5 shows quantification of a serial dilution of HPV16 DNA in human plasma.

FIG. 6 shows analysis of various annealing/extension temperatures for amplification of DNA using HPV18 and HPV33 primers and probes.

FIG. 7 shows detection of a serial dilution of HPV33 and HPV18 DNA.

FIGS. 8A and 8B show quantification of a serial dilution of HPV33 and HPV18 DNA.

FIG. 9 shows quantification of a serial dilution of HPV33 and HPV18 DNA

FIG. 10 shows detection of a serial dilution of HPV33 and HPV18 DNA in human plasma.

FIGS. 11A and 11B show quantification of a serial dilution of HPV33 and HPV18 DNA in human plasma.

FIG. 12 shows analysis of various annealing/extension temperatures for amplification of DNA using HPV35 and HPV45 primers and probes.

FIG. 13 shows detection of a serial dilution of HPV35 and HPV45 DNA.

FIGS. 14A and 14B show quantification of a serial dilution of HPV35 and HPV45 DNA.

FIG. 15 shows quantification of a serial dilution of HPV35 and HPV45 DNA.

FIG. 16 shows detection of a serial dilution of HPV35 and HPV45 DNA in human plasma.

FIGS. 17A and 17B show quantification of a serial dilution of HPV35 and HPV45 DNA in human plasma.

FIG. 18 shows analysis of various annealing/extension temperatures for amplification of DNA using HPV31 and HPV58 primers and probes.

FIG. 19 shows detection of a serial dilution of HPV31 and HPV58 DNA.

FIGS. 20A and 20B show quantification of a serial dilution of HPV31 and HPV58 DNA.

FIG. 21 shows quantification of a serial dilution of HPV31 and HPV58 DNA.

FIG. 22 shows detection of a serial dilution of HPV31 and HPV58 DNA in human plasma.

FIG. 23 shows quantification of a serial dilution of HPV31 and HPV58 DNA in human plasma.

FIG. 24 shows analysis of various annealing/extension temperatures for amplification of DNA using HPV52 and HPV59 primers and probes.

FIG. 25 shows detection of a serial dilution of HPV52 and HPV59 DNA.

FIGS. 26A and 26B show quantification of a serial dilution of HPV52 and HPV59 DNA.

FIG. 27 shows quantification of a serial dilution of HPV52 and HPV59 DNA.

FIG. 28 shows detection of a serial dilution of HPV52 and HPV59 DNA in human plasma.

FIGS. 29A and 29B show quantification of a serial dilution of HPV52 and HPV59 DNA in human plasma.

FIG. 30 shows analysis of various annealing/extension temperatures for amplification of DNA using HPV39 and HPV51 primers and probes.

FIG. 31 shows detection of a serial dilution of HPV39 and HPV51 DNA.

FIGS. 32A and 32B show quantification of a serial dilution of HPV39 and HPV51 DNA.

FIG. 33 shows quantification of a serial dilution of HPV39 and HPV51 DNA.

FIG. 34 shows detection of a serial dilution of HPV39 and HPV51 DNA in human plasma.

FIGS. 35A and 35B show quantification of a serial dilution of HPV39 and HPV51 DNA in human plasma.

FIG. 36 shows results from an analysis for potential cross reactivity between HPV types.

FIG. 37 shows results from an analysis for potential cross reactivity between HPV types.

FIG. 38 shows results from an analysis for potential cross reactivity between HPV types.

FIG. 39 shows results from an analysis for potential cross reactivity between HPV types.

FIG. 40 shows results from an analysis for potential cross reactivity between HPV types.

FIG. 41 shows detection of a serial dilution of HPV18 DNA in human plasma with or without ERV3 DNA.

FIG. 42 shows quantification of a serial dilution of HPV18 DNA with or without ERV3 DNA.

FIG. 43 shows detection of a serial dilution of HPV31 DNA in human plasma with or without ERV3 DNA.

FIG. 44 shows quantification of a serial dilution of HPV31 DNA with or without ERV3 DNA.

FIG. 45 shows detection of a serial dilution of HPV33 DNA in human plasma with or without ERV3 DNA.

FIG. 46 shows quantification of a serial dilution of HPV33 DNA with or without ERV3 DNA.

FIG. 47 shows detection of a serial dilution of HPV35 DNA in human plasma with or without ERV3 DNA.

FIG. 48 shows quantification of a serial dilution of HPV35 DNA with or without ERV3 DNA.

FIG. 49 shows detection of a serial dilution of HPV39 DNA in human plasma with or without ERV3 DNA.

FIG. 50 shows quantification of a serial dilution of HPV39 DNA with or without ERV3 DNA.

FIG. 51 shows detection of a serial dilution of HPV45 DNA in human plasma with or without ERV3 DNA.

FIG. 52 shows quantification of a serial dilution of HPV45 DNA with or without ERV3 DNA.

FIG. 53 shows detection of a serial dilution of HPV51 DNA in human plasma with or without ERV3 DNA.

FIG. 54 shows quantification of a serial dilution of HPV51 DNA with or without ERV3 DNA.

FIG. 55 shows detection of a serial dilution of HPV52 DNA in human plasma with or without ERV3 DNA.

FIG. 56 shows quantification of a serial dilution of HPV52 DNA with or without ERV3 DNA.

FIG. 57 shows detection of a serial dilution of HPV56 DNA in human plasma with or without ERV3 DNA.

FIG. 58 shows quantification of a serial dilution of HPV56 DNA with or without ERV3 DNA.

FIG. 59 shows detection of a serial dilution of HPV58 DNA in human plasma with or without ERV3 DNA.

FIG. 60 shows quantification of a serial dilution of HPV58 DNA with or without ERV3 DNA.

FIG. 61 shows detection of a serial dilution of HPV59 DNA in human plasma with or without ERV3 DNA.

FIG. 62 shows quantification of a serial dilution of HPV59 DNA with or without ERV3 DNA.

FIG. 63 shows detection of a serial dilution of HPV68 DNA in human plasma with or without ERV3 DNA.

FIG. 64 shows quantification of a serial dilution of HPV68 DNA with or without ERV3 DNA.

### DETAILED DESCRIPTION

Aspects of the present disclosure are based, in least in part, on the development of methods and compositions for detection and quantification of a high number (e.g., 6, 7, 8, 9, 10, 11, 12, or more) of different HPV types from purified DNA from subjects, including DNA from, for example, a cytology sample, a biopsy sample, or a cell-free (e.g., serum, plasma) sample. As described in embodiments of the disclosure, an HPV type-specific digital droplet polymerase chain reaction (ddPCR) assay has been developed with high sensitivity, specificity, accuracy, precision, and reproducibility for detection and quantification of up to 13 high-risk HPV types and a control in circulating cell-free DNA (cfDNA) in plasma or serum. The assay can also be used on DNA extracted from various body fluids, cytology specimens, and tumor tissues. This assay has utility in detection of HPV from various HPV-associated cancers, including anal, cervical, vaginal, penile, and head and neck cancers.

### I. Detection of HPV DNA

Aspects of the present disclosure are directed to methods for detection of HPV DNA from a sample. In particular aspects, disclosed herein are methods for detecting which types of a set of high-risk HPV types are present in a sample (i.e., detecting whether DNA from each of the types is present in a sample). Thus, embodiments of the disclosure comprise methods for determining whether DNA from 0, 1, 2, 3, 4, 5, 6, 7, 8, 9, 10, 11, 12, 13, or more types of a set of high-risk HPV types are present in a sample. In one example, a method of the disclosure identifies that DNA from HPV16 is present in a sample, and that no DNA from any of HPV18, HPV31, HPV33, HPV35, HPV39, HPV45, HPV51, HPV52, HPV58, HPV59, HPV56, or HPV68 is present in the sample. In this example, the amount of HPV16 DNA in the sample may also be quantified. In another example, a method of the disclosure may determine that DNA from HPV16 and DNA HPV18 are both present in the sample, and that no DNA from any of HPV31, HPV33, HPV35, HPV39, HPV45, HPV51, HPV52, HPV58, HPV59, HPV56, or HPV68 is present in the sample. In this example, the amount of HPV16 DNA and HPV18 in the sample may also be quantified. Any combination of one or more HPV types from a set of HPV types may be detected and quantified using the disclosed methods. Methods for detecting high-risk HPV types may be useful in, for example, identifying a subject as having or being at risk for developing an HPV-associated cancer, and for monitoring a subject for response to a treatment for an HPV-associated cancer.

In some embodiments, the disclosed methods comprise amplifying HPV DNA from a biological sample from a subject (e.g., blood sample, plasma sample, serum sample, etc.). Amplifying HPV DNA from a biological sample may comprise first extracting DNA from the biological sample followed by an amplification procedure to amplify the HPV DNA. In some embodiments, the HPV DNA is amplified using 2, 3, 4, 5, 6, 7, 8, 9, 10, 11, 12, or 13 primer pairs, each configured to amplify a different HPV type of a set of high-risk HPV types. In some embodiments, the HPV DNA is amplified using at least 6, 7, 8, 9, 10, 11, 12, or 13 such primer pairs, for example at least 6, 7, 8, 9, 10, 11, or 12 of the primer pairs listed in Table 1. In some embodiments, the HPV DNA is amplified using at least 7, 8, 9, 10, 11, or 12 primer pairs, such as at least 7, 8, 9, 10, 11, or 12 of the primer pairs listed in Table 1. In some embodiments, genomic DNA is amplified, using at least one primer pair, in addition to amplifying the HPV DNA. In some embodiments, a region of the ERV-3 gene is amplified using a primer pair (e.g., the ERV-3 primer pair of Table 1). Prior to amplification, the HPV DNA and/or genomic DNA may be separated into two or more wells, tubes, or the like and each subjected to emulsification. The emulsification process (methods for which are known to those skilled in the art and examples of which are provided in, for example, U.S. Pat. No. 9,089,844) may comprise partitioning the HPV DNA and/or genomic DNA into individual droplets within the wells or tubes, such that the HPV DNA and/or genomic DNA is separated into two or more sets of droplets with each set of droplets in a single well or tube. Each set of droplets may comprise one or more primer pairs for amplifying a region of HPV DNA and/or genomic DNA. Each set of droplets may comprise one primer pair of at least 6, 7, 8, 9, 10, 11, 12, 13, or 14 primer pairs. In some embodiments, each set of droplets comprises two primer pairs, such as any two primer pairs of the primer pairs listed in Table 1. In some embodiments, each set of droplets further comprises two probes, such as any two probes of the probes listed in Table 2. The DNA in each set of droplets may be amplified using the primer pairs, followed by detection using, for example, a droplet reader, thereby detecting which HPV types of a set of high-risk HPV types are present in a sample. Emulsification and droplet generation may be excluded from certain aspects of the disclosure.

Methods for detecting and/or quantifying the level of a target nucleic acid, for example, HPV DNA such as DNA from HPV16, HPV18, HPV31, HPV33, HPV35, HPV39, HPV45, HPV51, HPV52, HPV58, HPV59, HPV56, and HPV68, in a sample may involve the process of nucleic acid amplification, e.g., by polymerase chain reaction (PCR), ligase chain reaction (LCR), transcription-based amplification systems, (TAS) self-sustained sequence replication (3SR), nucleic acid sequence-based amplification (NASBA), strand displacement amplification (SDA) and branched DNA (bDNA) amplification, Q-Beta replicase, rolling circle replication, rolling circle amplification, or any other nucleic acid amplification method, followed by detection of the amplified molecules using any technique as would be appreciated by one of skill in the art. In some embodiments, the disclosed methods comprise digital droplet PCR. In some embodiments, the disclosed methods comprise quantitative PCR.

In some embodiments, the nucleic acid detection method may be carried out in partitions, droplets, or other micro-reaction chambers so that the detection method can be run in a highly parallelized manner. In some embodiments, droplets or partitions may contain either 0 or 1 copies of the HPV DNA region targeted for detection.

In embodiments involving emulsion PCR, a target nucleic acid or polynucleotide sequence is typically dispersed into droplets. In some embodiments, it is essential that the target nucleic acids be emulsified at a concentration where each droplet contains, on average, either one or zero copies of the target molecule. In the case of plasma or serum DNA isolated from patient blood samples, the appropriate dilution level can be recognized by assessing the abundance of a control genomic region and assuring that the frequency of positive micro-droplets remain less than 10% (<10%). The control genomic region (e.g., ERV3) detects human genomic (i.e., non-viral) DNA and serves as a quality control for the sample (since many negative samples will not have any positive signal in the assay), and will also be used to establish that the concentration of DNA fragments is appropriate.

Within each droplet, the principles of conventional PCR also apply, where the target molecule is amplified by reaction with at least one oligonucleotide primer or pair of oligonucleotide primers. The primers hybridize to a complementary region of the target nucleic acid and a DNA polymerase extends the primers to amplify the target sequence. Under conditions sufficient to provide polymerase-based nucleic acid amplification products, a nucleic acid fragment of one size dominates the reaction products (the target polynucleotide sequence which is the amplification product). The amplification cycle is repeated to increase the concentration of the single target nucleic acid or polynucleotide sequence within each droplet. The reaction can be performed in any thermal cycler commonly used for PCR.

Methods for setting up a PCR reaction are well known to those skilled in the art. Any known DNA polymerase, nucleoside triphosphate, buffers, additives/reaction enhancers and conditions for amplification (cycles of denaturation, annealing and polymerization) as would be appreciated by one of skill in the art may be used in the PCR reaction.

In some embodiments, the reaction includes a sequence-specific, hydrolysis probe that is conjugated to both a fluorescent molecule and a fluorescence-quenching molecule to the reaction mixture to enable the detection of successful amplification of the target molecule within each droplet. The chemical composition of specific probes may vary, but follow an established method for detecting synthesis-based nucleic acid amplification by those skilled in the art.

The preparation of an emulsion of the PCR reaction can be achieved in a variety of ways that are appreciated by those versed in the art. One effective methodology utilizes a fabricated microfluidic chip that mixes the aqueous PCR reaction with a lipid solution at controlled pressure to generate micro-droplets of uniform size. The disclosed method is applicable to any method for achieving a partitioned PCR reaction mixture that allows the simultaneous detection of two or more viral nucleic acid target molecules.

Following preparation of a PCR reaction mixture that has been appropriately emulsified or partitioned into droplets, the reaction mixture is subjected to primer extension reaction conditions, i.e., conditions that permit for polymerase mediated primer extension by addition of nucleotides to the end of the primer molecule using the template strand as a template. Cycles of denaturation, annealing and polymerization may be performed according to any conditions (e.g., number of cycles, temperatures and duration in time) that would be appreciated by one of skill in the art. Cycles of denaturation, annealing and polymerization may be performed using an automated device, typically known as a thermal cycler. Thermal cyclers that may be employed are described elsewhere herein as well as in U.S. Pat. Nos. 5,612,473; 5,602,756; 5,538,871; and 5,475,610.

In other embodiments, non-PCR based applications may be used to detect a target nucleic acid sequence, for example, where such target may be immobilized on a solid support. Methods of immobilizing a nucleic acid sequence on a solid support are known in the art and are described in Ausubel et al. Current Protocols in Molecular Biology, John Wiley and Sons, Inc. and in protocols provided by the manufacturers, e.g. for membranes: Pall Corporation, Schleicher & Schuell, for magnetic beads: Dynal, for culture plates: Costar, Nalgenunc, and for other supports.

Other nucleic acid amplification procedures will be appreciated by one of skill in the art, such as, but not limited to, LCR, TAS, 3SR, NASBA, SDA, bDNA, and isothermal amplification. The disclosed method is not limited to the use of amplification by PCR, but rather includes the use of any nucleic acid amplification methods or any other procedures which may be useful in amplification of the sequences for the detection and/or quantification of the presence of or expression of one or more of the particular nucleic acid sequences described herein.

Detection of the presence of target molecules from a sample is not particularly limited, and may be accomplished by any technique appreciated by one of skill in the art. In some embodiments, detection may include hybridization of the target molecule with a target-specific probe, such as a nucleic acid probe, linked to a fluorescent marker. In other embodiments, the marker may be a non-fluorescent marker. The nature of the marker, fluorescent or non-fluorescent, is not particularly limited and may be any marker or label as would be appreciated by of skill in the art.

The detection and distinguishing of partitions (droplets) that contain a target molecule from partitions that contain zero target molecules is a critical step in digital PCR. This can be achieved using a variety of established techniques. In some embodiments, droplets are analyzed individually using a microfluidic channel and a fluorescence detector. Alternatively, advanced microscopy techniques may be implemented to count positive and negative droplets. The disclosed methods may be embodied with any nucleic acid detection method.

### II. Nucleic Acid Assays

Aspects of the methods include assaying nucleic acids to detect and/or quantify HPV DNA. It is contemplated that a number of different assays could be employed to analyze nucleic acids. Such assays include, but are not limited to, nucleic amplification, polymerase chain reaction, quantitative PCR, RT-PCR, in situ hybridization, digital PCR, ddPCR (droplet digital PCR), nCounter (nanoString), BEAMing (Beads, Emulsions, Amplifications, and Magnetics) (Inostics), ARMS (Amplification Refractory Mutation Systems), RNA-Seq. TAm-Seg (Tagged-Amplicon deep sequencing), PAP (Pyrophosphorolysis-activation polymerization), next generation RNA sequencing, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a region of a target nucleic acid (e.g., HPV DNA). The term “primer” as used herein, describes any nucleic acid that is capable of priming the synthesis of a nascent nucleic acid in a template-dependent process and/or pairing with a single strand of a nucleic acid of the disclosure, or portion thereof. Typically, primers are oligonucleotides from ten to thirty nucleic acids in length, but longer sequences can be employed. Primers may be provided in double-stranded and/or single-stranded form, although the single-stranded form is preferred. Aspects of the disclosure are directed to one or more primer pairs. As used herein, a “primer pair” refers to a set of two primers, a “forward primer” (also “sense primer”) and a “reverse primer” (also “antisense primer”), having sequences which facilitate amplification of a region of a target nucleic acid (e.g., HPV DNA). Example primer pairs disclosed herein include those provided in Table 1. Various methods for use of primers for amplification of target nucleic acids are known to those skilled in the art.

TABLE 1
Example Primer Pairs

| Target | Forward primer | Reverse primer |
|---|---|---|
| HPV16 | GCACAGAGCTGCAAACAACT | AAAAGCAAAGTCATATACCTCACG |
| E6 | (SEQ ID NO: 1) | (SEQ ID NO: 2) |
| ERV-3 | GATAATTTCACACTAACCGCC | AGATGCTCTGACTTGATGGT |
| | (SEQ ID NO: 27) | (SEQ ID NO: 28) |
| HPV18 | GAAAGCTCAGCAGACGACCT | CACACCACGGACACACAAAG (SEQ |
| E7 | (SEQ ID NO: 3) | ID NO: 4) |
| HPV33 | GTTGTCACACTTGTAACACCAC | TGTGCCCATAAGTAGTTGCTGT |
| E7 | (SEQ ID NO: 5) | (SEQ ID NO: 6) |
| HPV35 | AGCAAAACCAGACACCTCCA | AGCAAAACCAGACACCTCCA (SEQ |
| E7 | (SEQ ID NO: 7) | ID NO: 8) |
| HPV45 | TTGCATTTGGAACCTCAGAA | TGCTTCATCGTTTTCCTCCT (SEQ ID |
| E7 | (SEQ ID NO: 9) | NO: 10) |
| HPV31 | GAGCACACAAGTAGATATTCGC | AGTAGAACAGTTGGGGCACA (SEQ |
| E7 | A (SEQ ID NO: 11) | ID NO: 12) |
| HPV58 | AGGCATGTGGATTTAAACAAAA | TTGTGTTTGTCTACGTCGGG (SEQ |
| E6 | GG (SEQ ID NO: 13) | ID NO: 14) |
| HPV52 | CAACTGACCTACACTGCTATGA | GCTTGTTCTGCTTGTCCATC (SEQ |
| E7 | (SEQ ID NO: 15) | ID NO: 16) |
| HPV59 | GTAGTAGAAACCTCGCAAGAC | GCACACAAAGGACACACAAAG |
| E7 | G (SEQ ID NO: 17) | (SEQ ID NO: 18) |
| HPV39 | AGGTGCATGTGTTGTCTGA | CCTGTATAGCTTCCTGCTATT (SEQ |
| E6 | (SEQ ID NO: 19) | ID NO: 20) |
| HPV51 | TATGCGTGACCAGCTACCAG | AACACCTGCAACACGGAGCT (SEQ |
| E7 | (SEQ ID NO: 21) | ID NO: 22) |
| HPV56 | ACAGCTCAGAGGATGAGGAT | TAGCTTGTCTAGCTTGCTGTG (SEQ |
| E7 | (SEQ ID NO: 23) | ID NO: 24) |
| HPV68 | CTTGTATGTCACGAGCAATTAG | TGTGACGCTGTTGTTCGTC (SEQ ID |
| E7 | (SEQ ID NO: 25) | NO: 26) |

The term “probe” as used herein, is meant to encompass any oligonucleotide having a sequence that will recognize and form a duplex with a complementary sequence in a target nucleic acid (e.g., a region of HPV DNA). In some embodiments, a probe contains, in addition to the nucleic acid sequence, one or more reporter molecules (e.g., fluorophores) for use in detection of a target nucleic acid sequence. In some embodiments, a probe comprises a fluorophore and a quencher, such that amplification of a target nucleic acid causes separation of the fluorophore from the quencher and resultant signal, thereby detecting amplification of the target nucleic acid. Example probes disclosed herein are provided in Table 2. Various methods for design and use of probes for detection of target nucleic acids are known to those skilled in the art, examples of which are disclosed in, for example, U.S. Pat. Nos. 7,241,596, 5,925,517, 6,214,979, 7,435,561, and US Patent Application Publication NO. 2005/0214809, each of which is incorporated by reference herein in its entirety.

TABLE 2
Example Probes

| Target | Probe Sequence | Fluorophore | Quencher |
|---|---|---|---|
| HPV16 | TGTGTACTGCAAGCAACAGTTACTGCG (SEQ | FAM | BHQ |
| E6 | ID NO: 29) | | |
| ERV-3 | TCTTCCCTCGAACCTGC (SEQ ID NO: 42) | HEX | BHQ |
| HPV18 | CGAGCATTCCAGCAGCTGTTTCTG (SEQ ID | FAM | BHQ |
| E7 | NO: 30) | | |
| HPV33 | TGTCAACAGTACAGCAAGTGACCTACGAA | HEX | BHQ |
| E7 | (SEQ ID NO: 31) | | |
| HPV35 | ACGTCCTGTTGTAAATGTGAGGCGA (SEQ ID | FAM | BHQ |
| E7 | NO: 32) | | |
| HPV45 | CCTGTTGACCTGTTGTGTTACGAGCA (SEQ ID | HEX | BHQ |
| E7 | NO: 33) | | |
| HPV31 | AGCTGTTAATGGGCTCATTTGGAATCG (SEQ | FAM | BHQ |
| E7 | ID NO: 34) | | |
| HPV58 | ATATTTCGGGTCGTTGGACAGGGC (SEQ ID | HEX | BHQ |
| E6 | NO: 35) | | |
| HPV52 | AGGTGACAGCTCAGATGAGGAGGA (SEQ ID | FAM | BHQ |
| E7 | NO: 36) | | |
| HPV59 | TGCGAGCCTTACAGCAGCTGTTTA (SEQ ID | HEX | BHQ |
| E7 | NO: 37) | | |
| HPV39 | AACCGCTGTGTCCAGCAGAAA (SEQ ID NO: 38) | FAM | BHQ |
| E6 | | | |
| HPV51 | CGTAGCCTGTCCAGCCCGTC (SEQ ID NO: 39) | HEX | BHQ |
| E7 | | | |
| HPV56 | TGAAGTAGACCATTTGCAGGAGCGG (SEQ ID | HEX | BHQ |
| E7 | NO: 40) | | |
| HPV68 | CATGCAGTTAATCACCACCAACATC (SEQ ID | FAM | BHQ |
| E7 | NO: 41) | | |

In some embodiments, a primer comprises at least 10 nucleotides. For instance, a primer can comprise at least or at most 10, 15, 20, 25, 50, 75, 100, 125, 150, 175, 200, 225, 250, 275, 300, 325, 350, or 400 nucleotides (or any range or value derivable therein). In some embodiments, a probe comprises at least 10 nucleotides. For instance, a probe can comprise at least or at most 10, 15, 20, 25, 50, 75, 100, 125, 150, 175, 200, 225, 250, 275, 300, 325, 350, or 400 nucleotides (or any range or value derivable therein).

In some embodiments, quantitative RT-PCR (such as TaqMan, ABI) is used for detecting and comparing the levels or abundance of nucleic acids in a sample (e.g., HPV DNA). The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In one embodiment, the PCR amplification utilizes one or more internal PCR standards. The internal standard may be an abundant housekeeping gene in the cell or it can specifically be GAPDH, GUSB and/or β-2 microglobulin. These standards may be used to normalize expression levels so that the expression levels of different gene products can be compared directly. A person of ordinary skill in the art would know how to use an internal standard to normalize expression levels.

In some embodiments, droplet digital PCR (ddPCR) is used for detecting and/or quantifying nucleic acids in a sample (e.g., HPV DNA). ddPCR methods may comprise partitioning of a sample into a population of partitions (e.g., microwells, droplets, etc.) and amplifying and detecting nucleic acid in each partition. In some embodiments, a sample is partitioned at a dilution such that each droplet contains, on average, either 0 or 1 copy of a given target nucleic acid. In this way, ddPCR can be used for detection and quantification of target nucleic acid (e.g., HPV DNA) with high sensitivity. Certain methods comprising use of ddPCR for detection, quantification, and analysis of target nucleic acids are described in, for example, U.S. Pat. Nos. 9,347,059 and 10,612,081, each of which are incorporated by reference herein in its entirety.

### A. Sequences

Sequences of primers and probes used in certain embodiments of the methods, systems, and kits of the present disclosure are provided in Table 3. In some embodiments, a primer of the disclosure comprises a sequence having at least or at most 90%, 91%, 92%, 93%, 94%, 95%, 96%, 97%, 98%, 99%, or 100% identity or homology with any of SEQ ID NOs:1-28. In some embodiments, a probe of the disclosure comprises a sequence having at least or at most 90%, 91%, 92%, 93%, 94%, 95%, 96%, 97%, 98%, 99%, or 100% identity or homology with any of SEQ ID NOs:29-42.

TABLE 3
Example Sequences

| | | SEQ |
|---|---|---|
| | | ID |
| Name | Sequence | NO: |
| HPV16 Forward Primer | GCACAGAGCTGCAAACAACT | 1 |
| HPV16 Reverse Primer | AAAAGCAAAGTCATATACCTCACG | 2 |
| HPV18 Forward Primer | GAAAGCTCAGCAGACGACCT | 3 |
| HPV18 Reverse Primer | CACACCACGGACACACAAAG | 4 |
| HPV33 Forward Primer | GTTGTCACACTTGTAACACCAC | 5 |
| HPV33 Reverse Primer | TGTGCCCATAAGTAGTTGCTGT | 6 |
| HPV35 Forward Primer | AGCAAAACCAGACACCTCCA | 7 |
| HPV35 Reverse Primer | AGCAAAACCAGACACCTCCA | 8 |
| HPV45 Forward Primer | TTGCATTTGGAACCTCAGAA | 9 |
| HPV45 Reverse Primer | TGCTTCATCGTTTTCCTCCT | 10 |
| HPV31 Forward Primer | GAGCACACAAGTAGATATTCGCA | 11 |
| HPV31 Reverse Primer | AGTAGAACAGTTGGGGCACA | 12 |
| HPV58 Forward Primer | AGGCATGTGGATTTAAACAAAAGG | 13 |
| HPV58 Reverse Primer | TTGTGTTTGTCTACGTCGGG | 14 |
| HPV52 Forward Primer | CAACTGACCTACACTGCTATGA | 15 |
| HPV52 Reverse Primer | GCTTGTTCTGCTTGTCCATC | 16 |
| HPV59 Forward Primer | GTAGTAGAAACCTCGCAAGACG | 17 |
| HPV59 Reverse Primer | GCACACAAAGGACACACAAAG | 18 |
| HPV39 Forward Primer | AGGTGCATGTGTTGTCTGA | 19 |
| HPV39 Reverse Primer | CCTGTATAGCTTCCTGCTATT | 20 |
| HPV51 Forward Primer | TATGCGTGACCAGCTACCAG | 21 |
| HPV51 Reverse Primer | AACACCTGCAACACGGAGCT | 22 |
| HPV56 Forward Primer | ACAGCTCAGAGGATGAGGAT | 23 |
| HPV56 Reverse Primer | TAGCTTGTCTAGCTTGCTGTG | 24 |
| HPV68 Forward Primer | CTTGTATGTCACGAGCAATTAG | 25 |
| HPV68 Reverse Primer | TGTGACGCTGTTGTTCGTC | 26 |
| ERV-3 Forward Primer | GATAATTTCACACTAACCGCC | 27 |
| ERV-3 Reverse Primer | AGATGCTCTGACTTGATGGT | 28 |
| HPV16 Probe | TGTGTACTGCAAGCAACAGTTACTGCG | 29 |
| HPV18 Probe | CGAGCATTCCAGCAGCTGTTTCTG | 30 |
| HPV33 Probe | TGTCAACAGTACAGCAAGTGACCTACGAA | 31 |
| HPV35 Probe | ACGTCCTGTTGTAAATGTGAGGCGA | 32 |
| HPV45 Probe | CCTGTTGACCTGTTGTGTTACGAGCA | 33 |
| HPV31 Probe | AGCTGTTAATGGGCTCATTTGGAATCG | 34 |
| HPV58 Probe | ATATTTCGGGTCGTTGGACAGGGC | 35 |
| HPV52 Probe | AGGTGACAGCTCAGATGAGGAGGA | 36 |
| HPV59 Probe | TGCGAGCCTTACAGCAGCTGTTTA | 37 |
| HPV39 Probe | AACCGCTGTGTCCAGCAGAAA | 38 |
| HPV51 Probe | CGTAGCCTGTCCAGCCCGTC | 39 |
| HPV56 Probe | TGAAGTAGACCATTTGCAGGAGCGG | 40 |
| HPV68 Probe | CATGCAGTTAATCACCACCAACATC | 41 |
| ERV-3 Probe | TCTTCCCTCGAACCTGC | 42 |

### III. Human Papilloma Virus

Aspects of the present disclosure involve detection and analysis of DNA from human papilloma virus (HPV). As used herein “human papilloma virus” or “HPV” refers to the small, non-enveloped, double-stranded DNA viruses that infects cutaneous and/or mucosal epithelium, as recognized in the art. As recognized herein, various HPV types have been associated with an increased risk for cancer. Such high-risk types include, for example, HPV16, HPV18, HPV31, HPV33, HPV35, HPV39, HPV45, HPV51, HPV52, HPV58, HPV59, HPV56, and HPV68. As used herein, HPV-associated malignancies (or HPV-associated cancers) include those of the head and neck (larynx, oral cavity, oropharynx, tonsils, and esophagus), respiratory tissue, skin, cervix, vulva, penis, and anus. In some embodiments, the HPV-associated cancer is cervical cancer. In some embodiments, the HPV-associated cancer is head and neck squamous cell carcinoma.

In some embodiments, methods of the present disclosure comprise detecting which types from a set of high-risk HPV types is present in a sample from a subject. A set of high-risk HPV types may comprise 2, 3, 4, 5, 6, 7, 8, 9, 10, 11, 12, or all of HPV16, HPV18, HPV31, HPV33, HPV35, HPV39, HPV45, HPV51, HPV52, HPV58, HPV59, HPV56, and HPV68. In some embodiments, a set of high-risk HPV types is a set of six of HPV16, HPV18, HPV31, HPV33, HPV35, HPV39, HPV45, HPV51, HPV52, HPV58, HPV59, HPV56, and HPV68. In some embodiments, a set of high-risk HPV types is a set of seven of HPV16, HPV18, HPV31, HPV33, HPV35, HPV39, HPV45, HPV51, HPV52, HPV58, HPV59, HPV56, and HPV68. In some embodiments, a set of high-risk HPV types is a set of eight of HPV16, HPV18, HPV31, HPV33, HPV35, HPV39, HPV45, HPV51, HPV52, HPV58, HPV59, HPV56, and HPV68. In some embodiments, a set of high-risk HPV types is a set of nine of HPV16, HPV18, HPV31, HPV33, HPV35, HPV39, HPV45, HPV51, HPV52, HPV58, HPV59, HPV56, and HPV68. In some embodiments, a set of high-risk HPV types is a set of ten of HPV16, HPV18, HPV31, HPV33, HPV35, HPV39, HPV45, HPV51, HPV52, HPV58, HPV59, HPV56, and HPV68. In some embodiments, a set of high-risk HPV types is a set of eleven of HPV16, HPV18, HPV31, HPV33, HPV35, HPV39, HPV45, HPV51, HPV52, HPV58, HPV59, HPV56, and HPV68. In some embodiments, a set of high-risk HPV types is a set of twelve of HPV16, HPV18, HPV31, HPV33, HPV35, HPV39, HPV45, HPV51, HPV52, HPV58, HPV59, HPV56, and HPV68. In some embodiments, a set of high-risk HPV types is a set of thirteen of HPV16, HPV18, HPV31, HPV33, HPV35, HPV39, HPV45, HPV51, HPV52, HPV58, HPV59, HPV56, and HPV68. A set of high-risk HPV types may comprise two or more of HPV16, HPV18, HPV31, HPV33, HPV35, HPV39, HPV45, HPV51, HPV52, HPV58, HPV59, HPV56, HPV68, in any combination. It is specifically contemplated that any one or more of HPV16, HPV18, HPV31, HPV33, HPV35, HPV39, HPV45, HPV51, HPV52, HPV58, HPV59, HPV56, and HPV68 may be excluded from embodiments of the disclosure. Detection of DNA from one or more types from the set of high-risk types may serve to diagnose the subject for an HPV-associated cancer or identify the subject at being at high risk for developing an HPV-associated cancer.

Methods of the present disclosure may comprise using detection of HPV DNA for monitoring of a response to a treatment for an HPV-associated cancer. For example, HPV DNA may be detected from a blood, plasma, or serum sample following administering a treatment for an HPV-associated cancer (e.g., a chemotherapy). The subject may be determined to be responsive to the treatment where a decrease in the amount of HPV DNA is identified. In some cases, a responsive subject is identified has having an initial increase in the amount of HPV DNA, corresponding with a release from tumor cells in response to a treatment, followed by a subsequent decrease in the amount of HPV DNA. In some cases, the subject may be determined to be nonresponsive to the treatment where an overall increase or no change in the amount of HPV DNA is identified.

### IV. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In other embodiments the sample may be obtained from any of the tissues provided herein that include but are not limited to non-cancerous or cancerous tissue and non-cancerous or cancerous tissue from the gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, plasma, serum, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be a cell-free sample. The biological sample may be obtained using any method known to the art that can provide a sample suitable for the analytical methods described herein. The sample may be obtained by non-invasive methods including but not limited to: scraping of the skin or cervix, swabbing of the check, blood collection, serum collection, plasma collection, saliva collection, urine collection, feces collection, and collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple cervical samples may be obtained for diagnosis by the methods described herein. In other cases, multiple samples, such as one or more samples from one tissue type (for example cervix) and one or more samples from another specimen (for example serum or plasma) may be obtained for diagnosis by the methods. In some cases, multiple samples such as one or more samples from one tissue type (e.g. cervix) and one or more samples from another specimen (e.g. serum, plasma, saliva) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a plasma sample, a serum sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a tumor or neoplasm or a suspected tumor or neoplasm. In some cases, the fine needle aspirate sampling procedure may be guided by the use of an ultrasound, X-ray, or other imaging device.

In some embodiments of the present methods, a molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, endocrinologist, or gynecologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

In some embodiments, a biological sample from a subject is a blood sample. In some embodiments, a biological sample from a subject is a plasma sample. In some embodiments, a biological sample from a subject is a serum sample. In some embodiments, a biological sample from a subject is a saliva sample. In some embodiments, a biological sample from a subject is a cell-free sample (i.e., a sample that does not contain whole, intact cells). A biological sample may comprise cell-free DNA (e.g., cell-free tumor DNA, cell-free HPV DNA, cell-free genomic DNA, etc.). A biological sample may be a cell-free sample comprising cell-free DNA. A biological sample may comprise both cells and cell-free DNA.

### V. Kits

Certain aspects of the present invention also concern kits containing compositions of the invention or compositions to implement methods of the invention. In some embodiments, kits can be used to evaluate one or more biomarkers. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, disclosed are kits for detection and/or quantification of HPV DNA from a sample.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any target nucleic acid identified herein (e.g., HPV DNA), which includes nucleic acid primers/primer sets and probes that are identical to or complementary to all or part of a target nucleic acid, which may include noncoding sequences as well as coding sequences. In some embodiments, disclosed are kits comprising at least, at most, or exactly 2, 3, 4, 5, 6, 7, 8, 9, 10, 11, 12, or 13 of the primer pairs of Table 1. In some embodiments, disclosed are kits comprising at least, at most, or exactly 2, 3, 4, 5, 6, 7, 8, 9, 10, 11, 12, or 13 of the probes of Table 2.

In certain aspects, negative and/or positive control nucleic acids, primers, and probes are included in some kit embodiments. In addition, a kit may include a sample that is a negative or positive control. In some embodiments, a control includes a nucleic acid that contains DNA from at least one high-risk HPV type. Various example control nucleic acids disclosed in embodiments of the present disclosure are provided in Table 4.

TABLE 4

| | | SEQ |
|---|---|---|
| | | ID |
| Name | Sequence | NO: |
| HPV16 E6 | AAAGTTACCACAGTTATGCACAGAGCTGCAAACAACT | 43 |
| control | ATACATGATATAATATTAGAATGTGTGTACTGCAAGC | |
| oligonucleotide | AACAGTTACTGCGACGTGAGGTATATGACTTTGCTTTT | |
| | CGGGATTTATGCATAGTATATAGAGATGGGA | |
| HPV18 E7 | TGTGTTGTAAGTGTGAAGCCAGAATTGAGCTAGTAGT | 44 |
| control | AGAAAGCTCAGCAGACGACCTTCGAGCATTCCAGCAG | |
| oligonucleotide | CTGTTTCTGAACACCCTGTCCTTTGTGTGTCCGTGGTG | |
| | TGCATCCCAGCAGTAAGCAACAATGGCTGATCCAGAA | |
| | GGT | |
| HPV33 E7 | TCTACACTTCGTTTGTGTGTACAGAGCACACAAGTAG | 45 |
| control | ATATTCGCATATTGCAAGAGCTGTTAATGGGCTCATTT | |
| oligonucleotide | GGAATCGTGTGCCCCAACTGTTCTACTAGACTGTAACT | |
| | ACAATGGCTGATCCAGCAGGTACAGATG | |
| HPV35 E7 | ACAACCAGCCACAGCTGATTACTACATTGTAACCTGTT | 46 |
| control | GTCACACTTGTAACACCACAGTTCGTTTATGTGTCAAC | |
| oligonucleotide | AGTACAGCAAGTGACCTACGAACCATACAGCAACTAC | |
| | TTATGGGCACAGTGAATATTGTGTGCCCTA | |
| HPV45 E7 | ATTGACGGTCCAGCTGGACAAGCAAAACCAGACACCT | 47 |
| control | CCAATTATAATATTGTAACGTCCTGTTGTAAATGTGAG | |
| oligonucleotide | GCGACACTACGTCTGTGTGTACAGAGCACACACATTG | |
| | ACATACGTAAATTGGAAGATTTATTAATGGGCACATT | |
| | TGGA | |
| HPV31 E7 | TAATACAAAGTTATATAATTTATTAATAAGGTGCATGT | 48 |
| control | GTTGTCTGAAACCGCTGTGTCCAGCAGAAAAATTAAG | |
| oligonucleotide | ACACCTAAATAGCAAACGAAGATTTCATAAAATAGCA | |
| | GGAAGCTATACAGGACAGTGTCGACGGTGCTGGA | |
| HPV58 E6 | AACACTGCAAGAAATTGTATTGCATTTGGAACCTCAG | 49 |
| control | AATGAATTAGATCCTGTTGACCTGTTGTGTTACGAGCA | |
| oligonucleotide | ATTAAGCGAGTCAGAGGAGGAAAACGATGAAGCAGA | |
| | TGGCGTTAGTCATGCACAACTACCAGCCCGACGAGCC | |
| | GAAC | |
| HPV52 E7 | ATTTGACAGCTCAGAGGAGGAGGATGAAGTAGATAAT | 50 |
| control | ATGCGTGACCAGCTACCAGAAAGACGGGCTGGACAG | |
| oligonucleotide | GCTACGTGTTACAGAATTGAAGCTCCGTGTTGCAGGT | |
| | GTTCAAGTGTAGTACAACTGGCAGTGGAAAG | |
| HPV59 E7 | TATATATTAGATCTGCAACCTGAAACAACTGACCTAC | 51 |
| control | ACTGCTATGAGCAATTAGGTGACAGCTCAGATGAGGA | |
| oligonucleotide | GGATACAGATGGTGTGGACCGGCCAGATGGACAAGC | |
| | AGAACAAGCCACAAGCAATTACTACATTGTGACA | |
| HPV39 E6 | CATTGTGTCCACAAGAAAAAAAAAGGCATGTGGATTT | 52 |
| control | AAACAAAAGGTTTCATAATATTTCGGGTCGTTGGACA | |
| oligonucleotide | GGGCGCTGTGCAGTGTGTTGGAGACCCCGACGTAGAC | |
| | AAACACAAGTGTAACCTGTAACAACGCCATGAGAGGA | |
| | AACA | |
| HPV51 E7 | GTGTGTTGTAAGTGTAATAATCAACTTCAGCTAGTAGT | 53 |
| control | AGAAACCTCGCAAGACGGATTGCGAGCCTTACAGCAG | |
| oligonucleotide | CTGTTTATGGACACACTATCCTTTGTGTGTCCTTTGTGT | |
| | GCAGCAAACCAGTAACCTGCAATGGC | |
| HPV56 E7 | TGAGCAATTGGACAGCTCAGAGGATGAGGATGAGGAT | 54 |
| control | GAAGTAGACCATTTGCAGGAGCGGCCACAGCAAGCTA | |
| oligonucleotide | GACAAGCTAAACAACATACGTGTTACCTAATACACGT | |
| | ACCTTGTTGTGAGTGTAAGTTTGTGGTGCAGTTGGA | |
| HPV68 E7 | GACCTTGTATGTCACGAGCAATTAGGAGATTCAGACG | 55 |
| control | ATGAAATAGATGAACCCGACCATGCAGTTAACCACCA | |
| oligonucleotide | CCAACATCAACTACTAGCCAGACGGGACGAACAACAG | |
| | CGTCACACAATTCAGTGTACGTGTTGTAAGTGTAACA | |
| | ACCTACTGCAACTAGTAGTAGAAGCGTCGCGG | |

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample.

Any embodiment of the disclosure involving a specific nucleic acid by name is contemplated also to cover embodiments involving nucleic acids whose sequences are at least 80, 81, 82, 83, 84, 85, 86, 87, 88, 89, 90, 91, 92, 93, 94, 95, 96, 97, 98, 99% identical (or any range or value derivable therein) to the sequence of the specified nucleic acid.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Development and Optimization of HPV16/ERV3 Amplification and Detection

Primers and probes for detection of human papillomavirus type 16 (HPV16) and endogenous retrovirus group 3 (ERV3; also “ERV-3”) were designed. ERV3 served to measure of human genomic DNA. Primer and probe sequences are provided in Table 5.

TABLE 5

| | | SEQ | | HPV |
|---|---|---|---|---|
| | | ID | Amplicon | gene |
| Name | Sequence | NO: | size (bp) | detected |
| HPV16 | GCACAGAGCTGCAAACAACT | 1 | 95 | E6 |
| forward | | | | |
| primer | | | | |
| HPV16 | AAAAGCAAAGTCATATACCTCACG | 2 | | |
| reverse | | | | |
| primer | | | | |
| HPV16 | TGTGTACTGCAAGCAACAGTTACTGCG | 29 | | |
| probe | | | | |
| (FAM- | | | | |
| labeled | | | | |
| w/ BHQ) | | | | |
| ERV3 | GATAATTTCACACTAACCGCC | 27 | 58 | — |
| forward | | | | |
| primer | | | | |
| ERV3 | AGATGCTCTGACTTGATGGT | 28 | | |
| reverse | | | | |
| primer | | | | |
| ERV3 | TCTTCCCTCGAACCTGC | 42 | | |
| probe | | | | |
| (HEX- | | | | |
| labeled | | | | |
| w/ BHQ) | | | | |

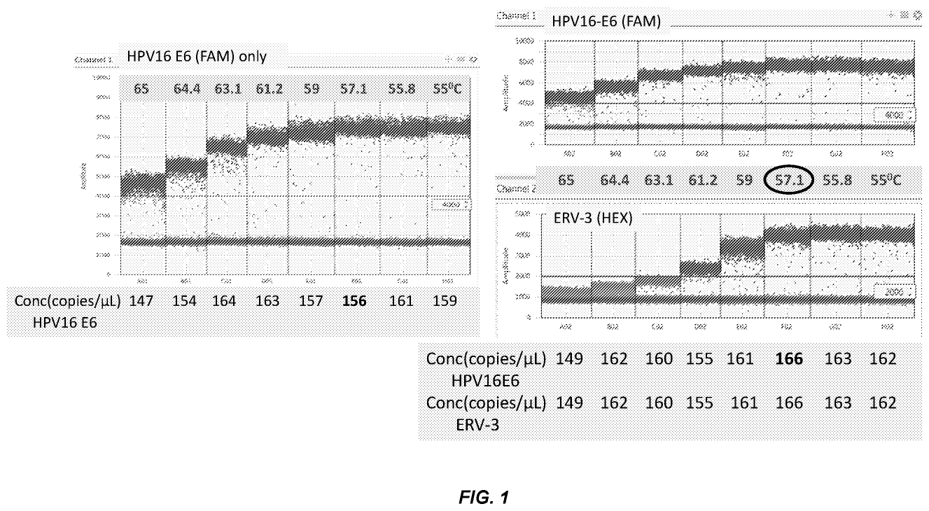

Amplification and detection with HPV16 and ERV3 primers and probes was tested at various annealing/extension temperatures from 55° C.-65° C., (FIG. 1) using nucleotide fragments comprising the amplicon sequences. 57.1° C. was identified as the best temperature for detection of both targets.

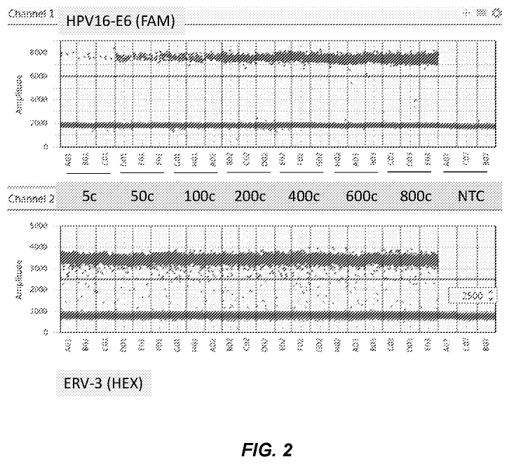

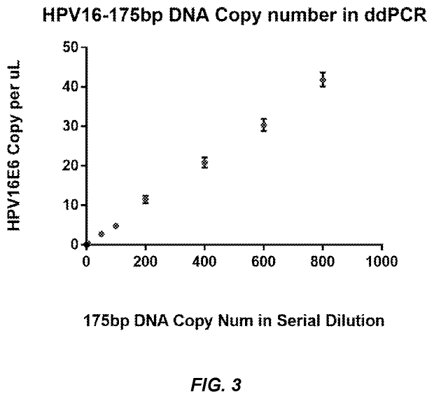

Amplification and detection with HPV16 and ERV3 primers and probes was tested using HPV16 fragment diluted from 800 copies down to 5 copies (FIGS. 2 and 3). The limit of detection (LoD) was determined to be 5 copies of HPV16 DNA per reaction.

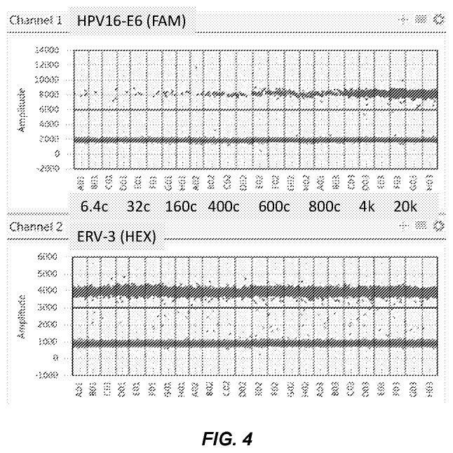

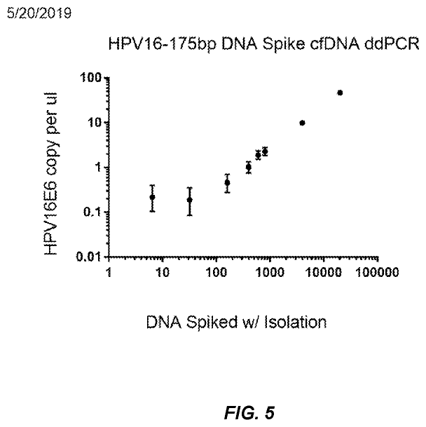

Amplification and detection with HPV16 and ERV3 primers and probes was tested using HPV16 fragment diluted from 20,000 (20 k) copies down to 6.4 copies in plasma from non-HPV infected individuals. DNA was extracted from the spiked plasma samples and analyzed using the HPV16 and ERV3 primers and probes via droplet digital PCR (FIGS. 4 and 5). The limit of quantification (LoQ) in plasma was determined to be 16 copies per ml of plasma.

### Example 2—Development and Optimization of HPV18/HPV33 Amplification and Detection

Primers and probes for detection of human papillomavirus type 18 (HPV18) and human papillomavirus type 33 (HPV33) were designed. Primer and probe sequences are provided in Table 6.

TABLE 6

| | | SEQ | | HPV |
|---|---|---|---|---|
| | | ID | Amplicon | gene |
| Name | Sequence | NO: | size (bp) | detected |
| HPV18 | GAAAGCTCAGCAGACGACCT | 3 | 76 | E7 |
| forward | | | | |
| primer | | | | |
| HPV18 | CACACCACGGACACACAAAG | 4 | | |
| reverse | | | | |
| primer | | | | |
| HPV18 | CGAGCATTCCAGCAGCTGTTTCTG | 30 | | |
| probe | | | | |
| (FAM- | | | | |
| labeled | | | | |
| w/ BHQ) | | | | |
| HPV33 | GTTGTCACACTTGTAACACCAC | 5 | 89 | E7 |
| forward | | | | |
| primer | | | | |
| HPV33 | TGTGCCCATAAGTAGTTGCTGT | 6 | | |
| reverse | | | | |
| primer | | | | |
| HPV33 | TGTCAACAGTACAGCAAGTGACCTACGAA | 31 | | |
| probe | | | | |
| (HEX- | | | | |
| labeled | | | | |
| w/ BHQ) | | | | |

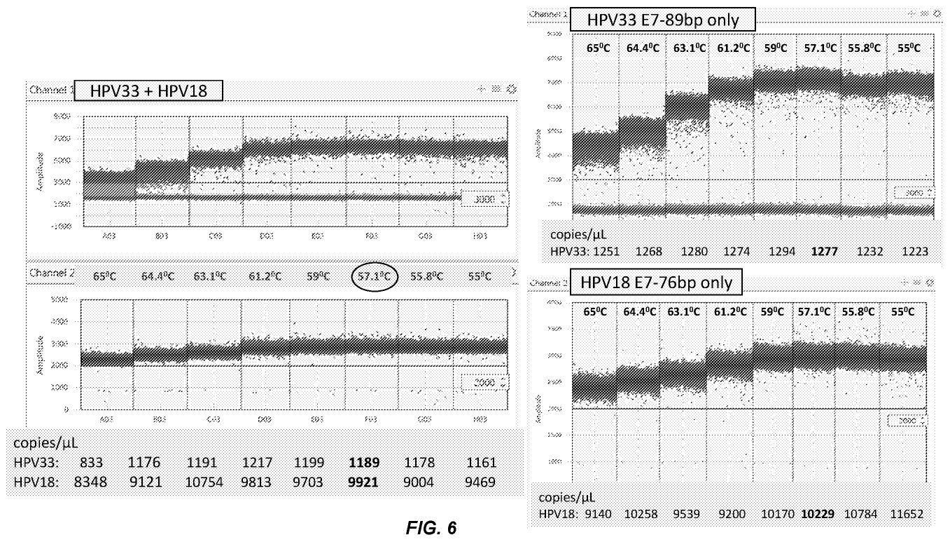

Amplification and detection with HPV18 and HPV33 primers and probes was tested at various annealing/extension temperatures from 55° C.-65° C., (FIG. 6) using nucleotide fragments comprising the amplicon sequences. 57.1° C. was identified as the best temperature for detection of both targets.

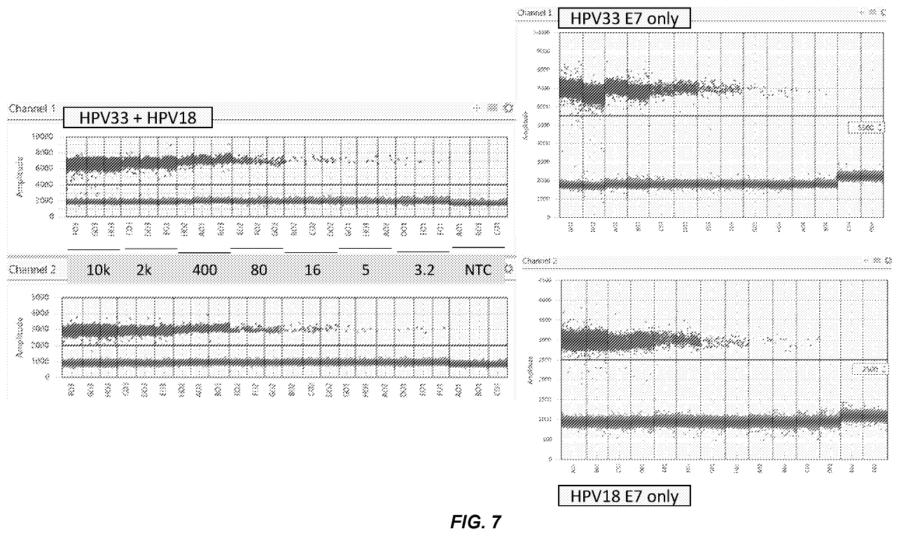

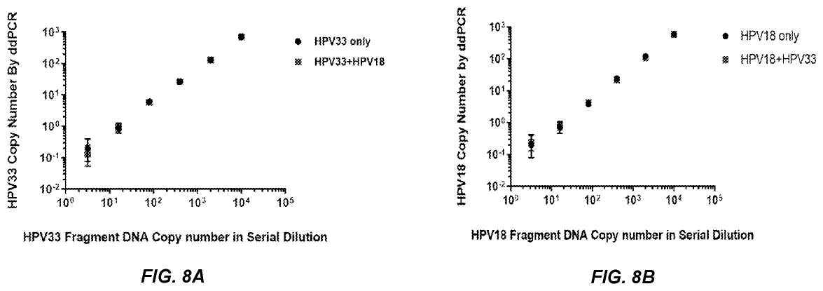

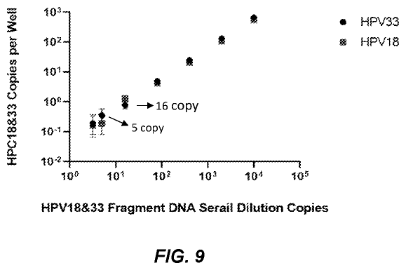

Amplification and detection with HPV18 and HPV33 primers and probes was tested using HPV18 and HPV33 fragments diluted from 10,000 (10 k) copies down to 3.2 copies (FIGS. 7-9). The limit of detection (LoD) was determined to be 5 copies of HPV18 DNA and 5 copies of HPV33 DNA per reaction.

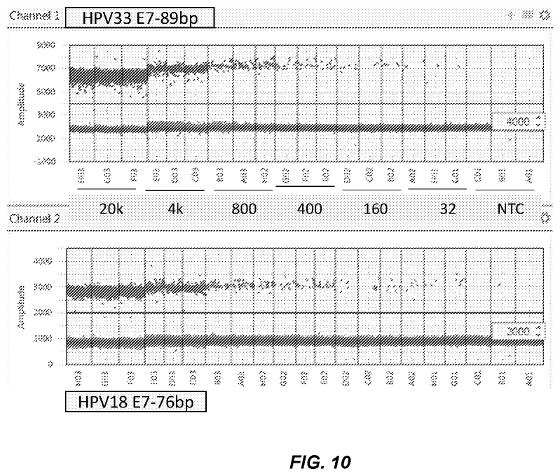

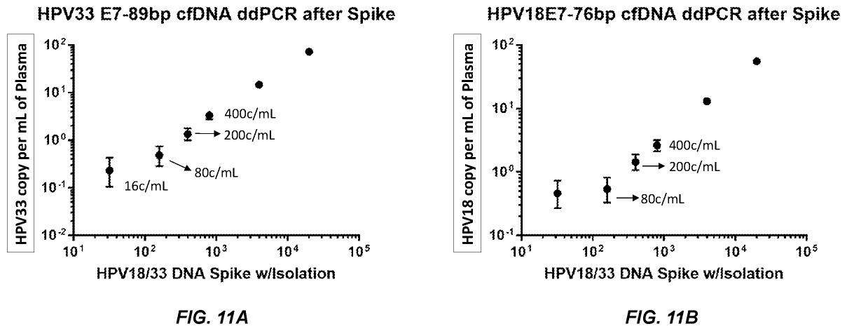

Amplification and detection with HPV18 and HPV33 primers and probes was tested using HPV18 and HPV33 fragments diluted from 20,000 (20 k) copies down to 32 copies in plasma from non-HPV infected individuals. DNA was extracted from the spiked plasma samples and analyzed using the HPV18 and HPV33 primers and probes via droplet digital PCR (FIGS. 10-11B). The limit of quantification (LoQ) in plasma was determined to be 16 copies per ml of plasma for both HPV18 and HPV33.

### Example 3—Development and Optimization of HPV35/HPV45 Amplification and Detection

Primers and probes for detection of human papillomavirus type 35 (HPV35) and human papillomavirus type 45 (HPV45) were designed. Primer and probe sequences are provided in Table 7.

TABLE 7

| | | | | HPV |
|---|---|---|---|---|
| | | SEQ | Amplicon | gene |
| Name | Sequence | ID NO: | size (bp) | detected |
| HPV35 | AGCAAAACCAGACACCTCCA | 7 | 96 | E7 |
| forward | | | | |
| primer | | | | |
| HPV35 | AGCAAAACCAGACACCTCCA | 8 | | |
| reverse | | | | |
| primer | | | | |
| HPV35 | ACGTCCTGTTGTAAATGTGAGGCGA | 32 | | |
| probe | | | | |
| (FAM- | | | | |
| labeled | | | | |
| w/ BHQ) | | | | |
| HPV45 | TTGCATTTGGAACCTCAGAA | 9 | 90 | E7 |
| forward | | | | |
| primer | | | | |
| HPV45 | TGCTTCATCGTTTTCCTCCT | 10 | | |
| reverse | | | | |
| primer | | | | |
| HPV45 | CCTGTTGACCTGTTGTGTTACGAGCA | 33 | | |
| probe | | | | |
| (HEX- | | | | |
| labeled | | | | |
| w/ BHQ) | | | | |

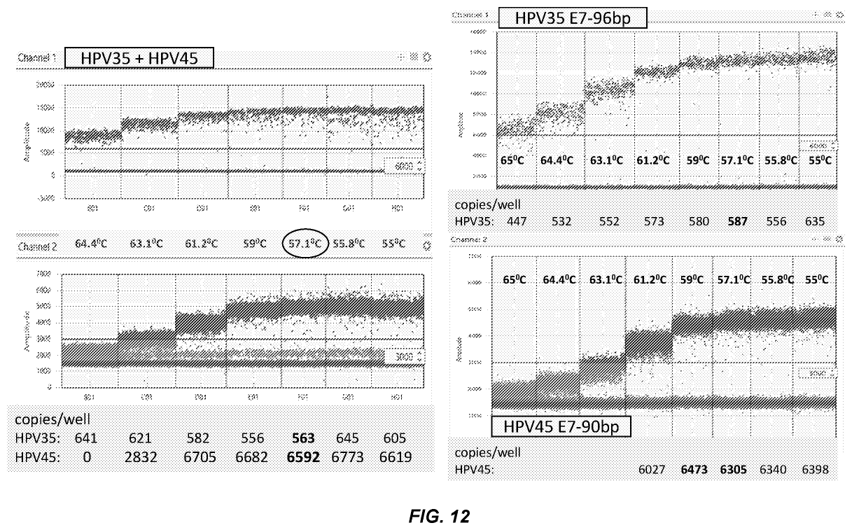

Amplification and detection with HPV35 and HPV45 primers and probes was tested at various annealing/extension temperatures from 55° C.-65° C., (FIG. 12) using nucleotide fragments comprising the amplicon sequences. 57.1° C. was identified as the best temperature for detection of both targets.

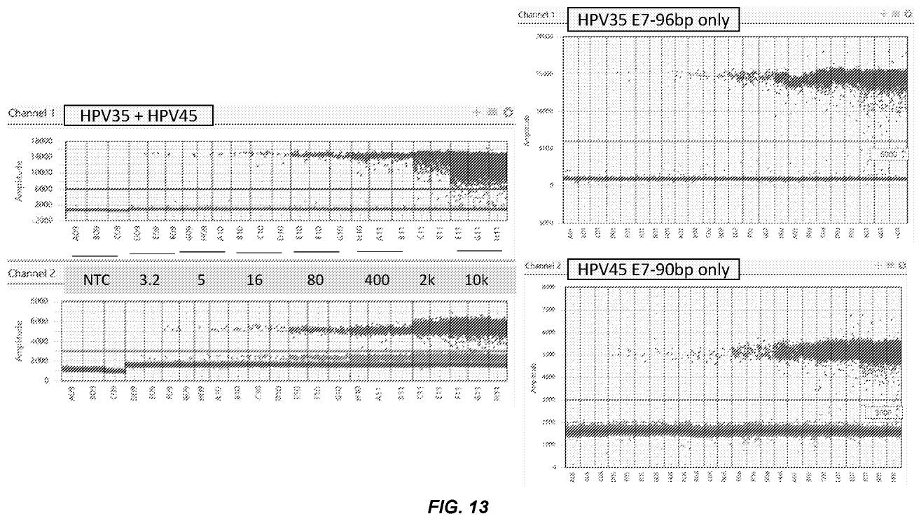

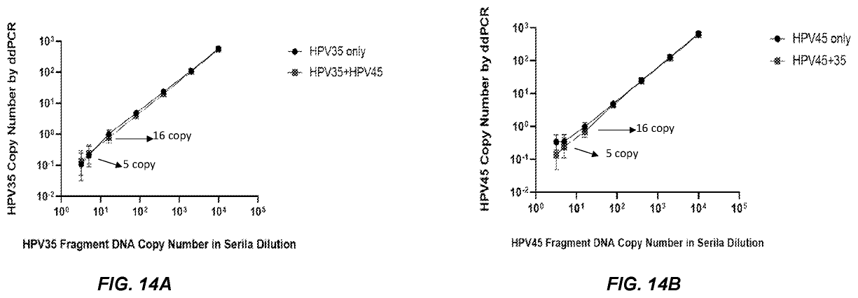

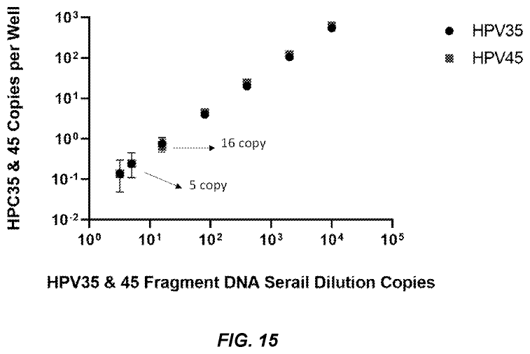

Amplification and detection with HPV35 and HPV45 primers and probes was tested using HPV35 and HPV45 fragments diluted from 10,000 (10 k) copies down to 3.2 copies (FIGS. 13-15). The limit of detection (LoD) was determined to be 5 copies of HPV35 DNA and 5 copies of HPV45 DNA per reaction.

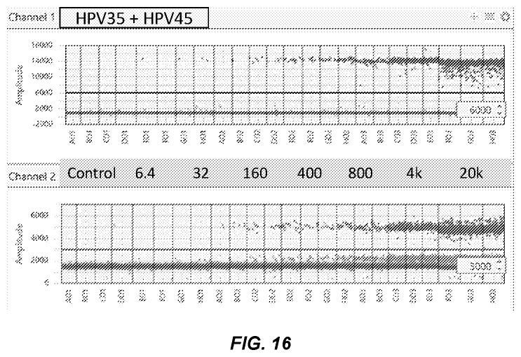

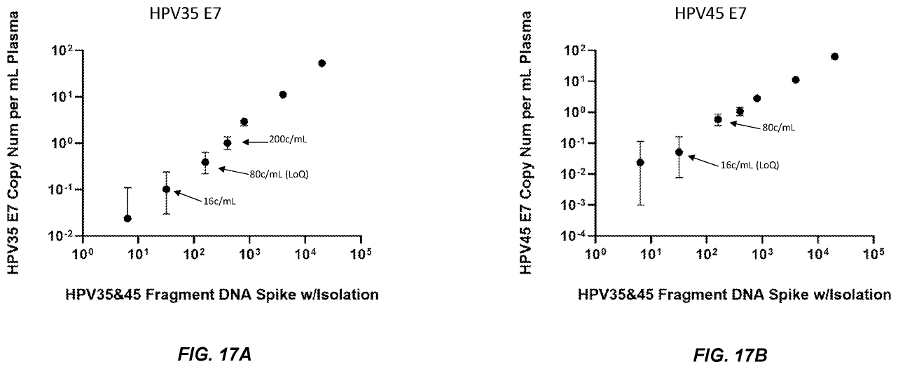

Amplification and detection with HPV35 and HPV45 primers and probes was tested using HPV35 and HPV45 fragments diluted from 20,000 (20 k) copies down to 6.4 copies in plasma from non-HPV infected individuals. DNA was extracted from the spiked plasma samples and analyzed using the HPV35 and HPV45 primers and probes via droplet digital PCR (FIGS. 16-17B). The limit of quantification (LoQ) in plasma was determined to be 16 copies per ml of plasma for HPV35 and 16 copies per ml of plasma for HPV45.

### Example 4—Development and Optimization of HPV31/HPV58 Amplification and Detection

Primers and probes for detection of human papillomavirus type 31 (HPV31) and human papillomavirus type 58 (HPV58) were designed. Primer and probe sequences are provided in Table 8.

TABLE 8

| | | | | HPV |
|---|---|---|---|---|
| | | SEQ | Amplicon | gene |
| Name | Sequence | ID NO: | size (bp) | detected |
| HPV31 | GAGCACACAAGTAGATATTCGCA | 11 | 79 | E7 |
| forward | | | | |
| primer | | | | |
| HPV31 | AGTAGAACAGTTGGGGCACA | 12 | | |
| reverse | | | | |
| primer | | | | |
| HPV31 | AGCTGTTAATGGGCTCATTTGGAATCG | 34 | | |
| probe | | | | |
| (FAM- | | | | |
| labeled | | | | |
| w/ BHQ) | | | | |
| HPV58 | AGGCATGTGGATTTAAACAAAAGG | 13 | 96 | E6 |
| forward | | | | |
| primer | | | | |
| HPV58 | TTGTGTTTGTCTACGTCGGG | 14 | | |
| reverse | | | | |
| primer | | | | |
| HPV58 | ATATTTCGGGTCGTTGGACAGGGC | 35 | | |
| probe | | | | |
| (HEX- | | | | |
| labeled | | | | |
| w/ BHQ) | | | | |

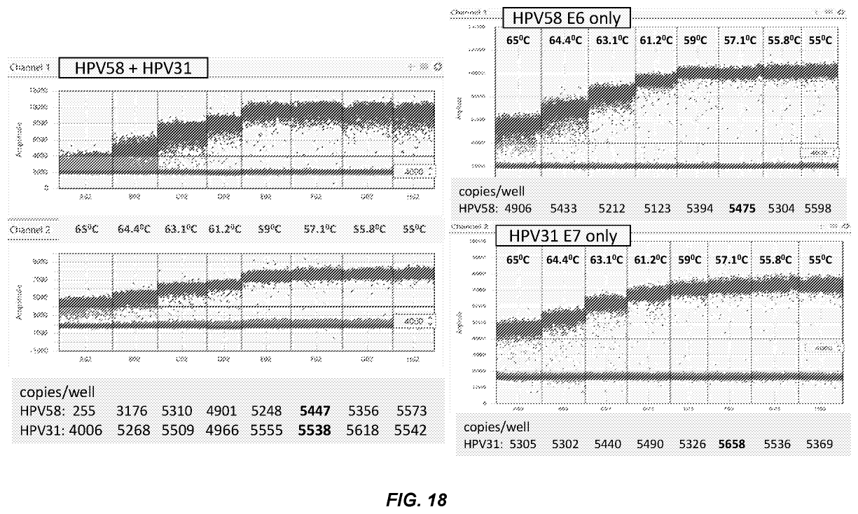

Amplification and detection with HPV31 and HPV58 primers and probes was tested at various annealing/extension temperatures from 55° C.-65° C., (FIG. 18) using nucleotide fragments comprising the amplicon sequences. 57.1° C. was identified as the best temperature for detection of both targets.

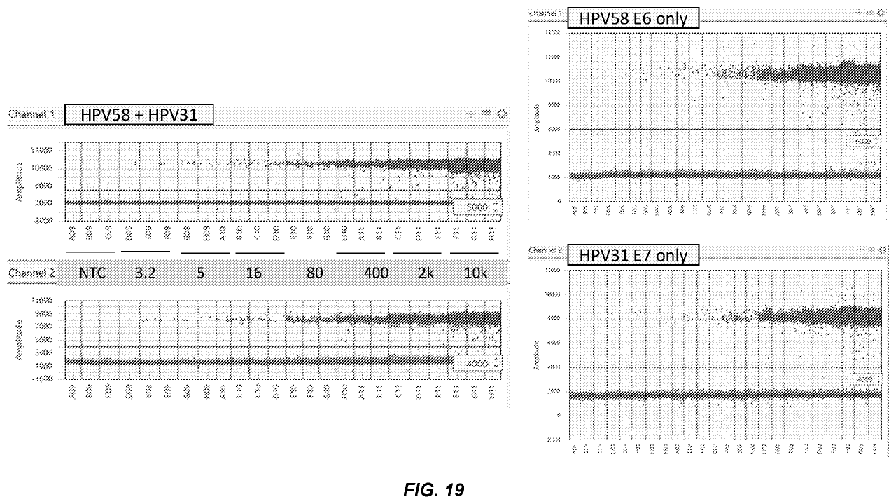

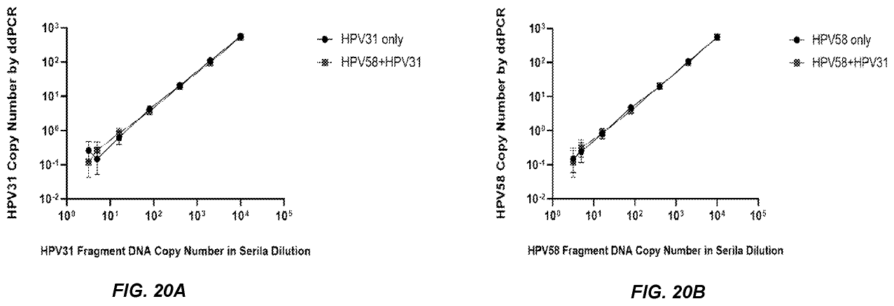

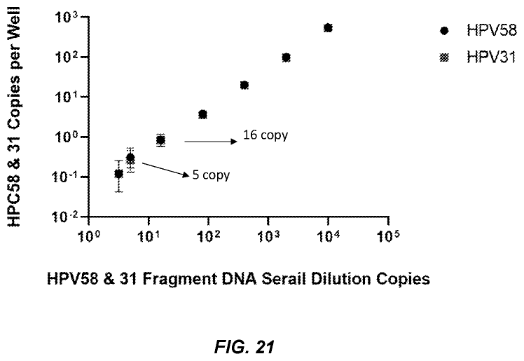

Amplification and detection with HPV31 and HPV58 primers and probes was tested using HPV31 and HPV58 fragments diluted from 10,000 (10 k) copies down to 3.2 copies (FIGS. 19-21). The limit of detection (LoD) was determined to be 5 copies of HPV31 DNA and 5 copies of HPV58 DNA per reaction.

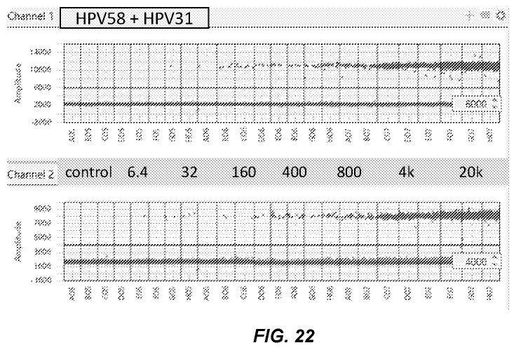

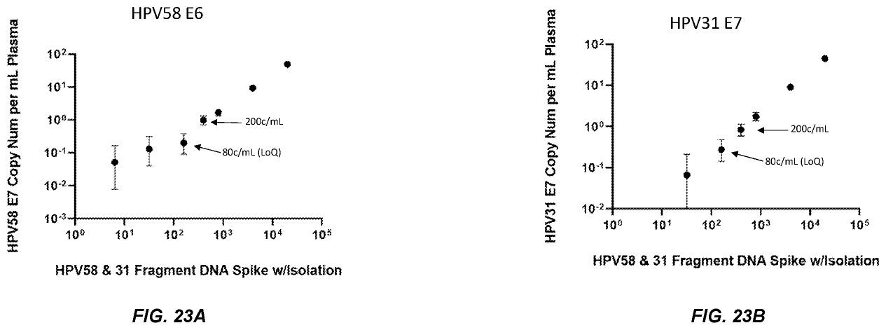

Amplification and detection with HPV31 and HPV58 primers and probes was tested using HPV31 and HPV58 fragments diluted from 20,000 (20 k) copies down to 6.4 copies in plasma from non-HPV infected individuals. DNA was extracted from the spiked plasma samples and analyzed using the HPV31 and HPV58 primers and probes via droplet digital PCR (FIGS. 22-23B). The limit of quantification (LoQ) in plasma was determined to be 16 copies per ml of plasma for both HPV31 and HPV58.

### Example 5—Development and Optimization of HPV52/HPV59 Amplification and Detection

Primers and probes for detection of human papillomavirus type 52 (HPV52) and human papillomavirus type 59 (HPV59) were designed. Primer and probe sequences are provided in Table 9.

TABLE 9

| | | | | HPV |
|---|---|---|---|---|
| | | SEQ ID | Amplicon | gene |
| Name | Sequence | NO: | size (bp) | detected |
| HPV52 | CAACTGACCTACACTGCTATGA | 15 | 94 | E7 |
| forward | | | | |
| primer | | | | |
| HPV52 | GCTTGTTCTGCTTGTCCATC | 16 | | |
| reverse | | | | |
| primer | | | | |
| HPV52 | AGGTGACAGCTCAGATGAGGAGGA | 36 | | |
| probe | | | | |
| (FAM- | | | | |
| labeled | | | | |
| w/ BHQ) | | | | |
| HPV59 | GTAGTAGAAACCTCGCAAGACG | 17 | 85 | E7 |
| forward | | | | |
| primer | | | | |
| HPV59 | GCACACAAAGGACACACAAAG | 18 | | |
| reverse | | | | |
| primer | | | | |
| HPV59 | TGCGAGCCTTACAGCAGCTGTTTA | 37 | | |
| probe | | | | |
| (HEX- | | | | |
| labeled | | | | |
| w/ BHQ) | | | | |

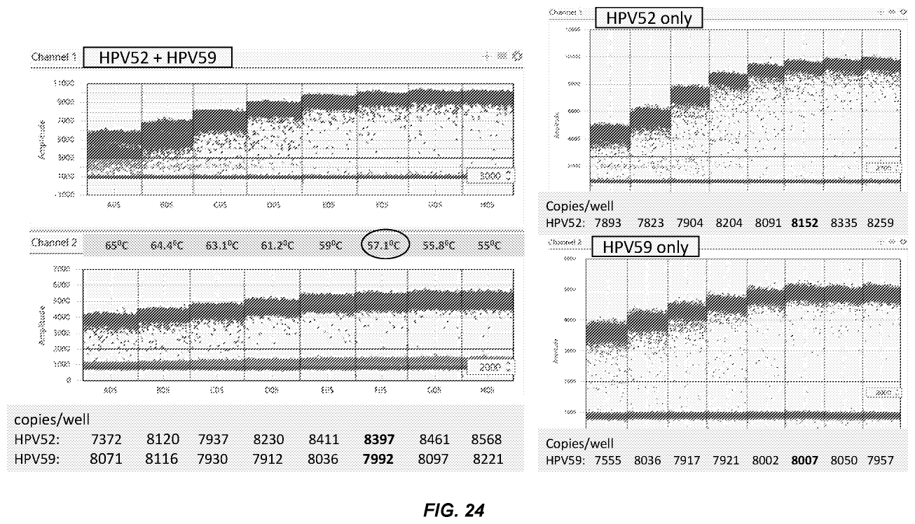

Amplification and detection with HPV52 and HPV59 primers and probes was tested at various annealing/extension temperatures from 55° C.-65° C., (FIG. 24) using nucleotide fragments comprising the amplicon sequences. 57.1° C. was identified as the best temperature for detection of both targets.

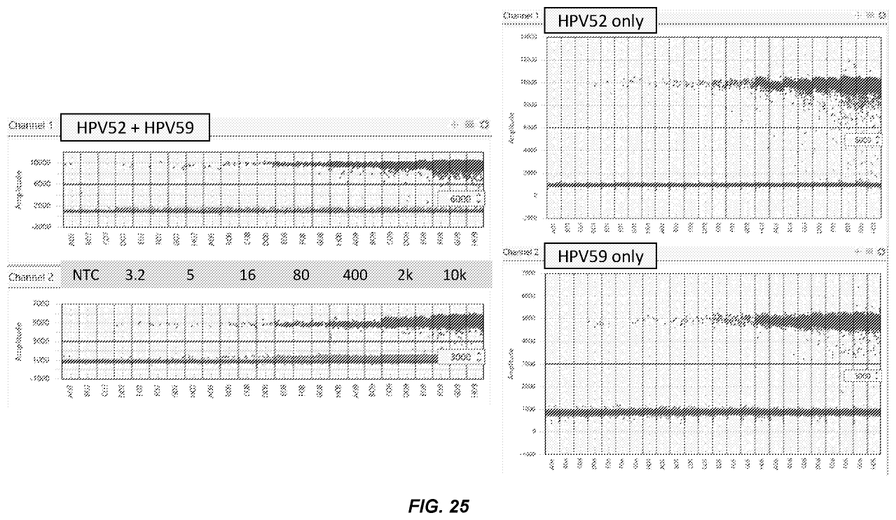

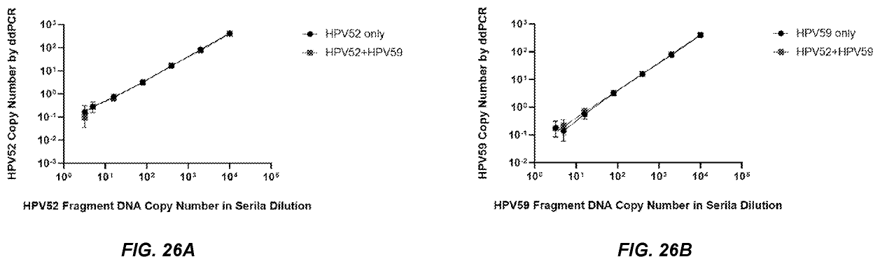

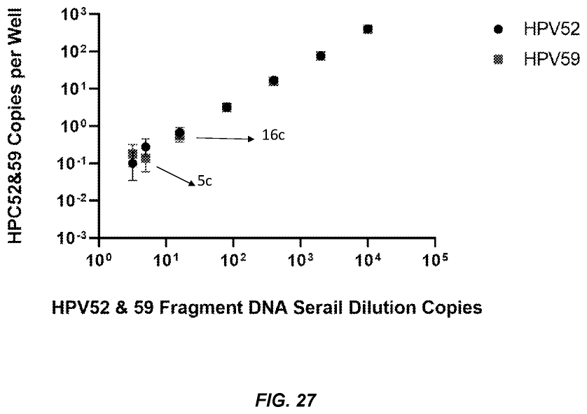

Amplification and detection with HPV52 and HPV59 primers and probes was tested using HPV52 and HPV59 fragments diluted from 10,000 (10 k) copies down to 3.2 copies (FIGS. 25-27). The limit of detection (LoD) was determined to be 5 copies of HPV52 DNA and 5 copies of HPV59 DNA per reaction.

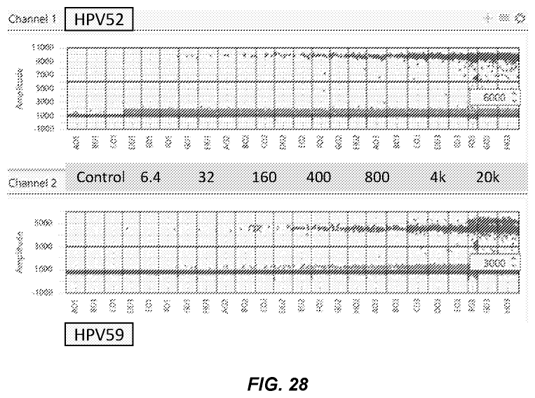

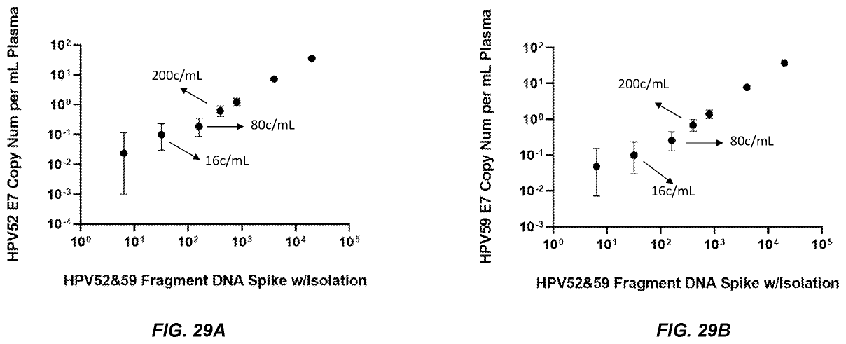

Amplification and detection with HPV52 and HPV59 primers and probes was tested using with HPV52 and HPV59 fragments diluted from 20,000 (20 k) copies down to 6.4 copies in plasma from non-HPV infected individuals. DNA was extracted from the spiked plasma samples and analyzed using the with HPV52 and HPV59 primers and probes via droplet digital PCR (FIGS. 28-29B). The limit of quantification (LoQ) in plasma was determined to be 16 copies per ml of plasma for both HPV52 and HPV59.

### Example 6—Development and Optimization of HPV39/HPV51 Amplification and Detection

Primers and probes for detection of human papillomavirus type 39 (HPV39) and human papillomavirus type 51 (HPV51) were designed. Primer and probe sequences are provided in Table 10.

TABLE 10

| | | | | HPV |
|---|---|---|---|---|
| | | SEQ ID | Amplicon | gene |
| Name | Sequence | NO: | size (bp) | detected |
| HPV39 | AGGTGCATGTGTTGTCTGA | 19 | 98 | E6 |
| forward | | | | |
| primer | | | | |
| HPV39 | CCTGTATAGCTTCCTGCTATT | 20 | | |
| reverse | | | | |
| primer | | | | |
| HPV39 | AACCGCTGTGTCCAGCAGAAA | 38 | | |
| probe | | | | |
| (FAM- | | | | |
| labeled | | | | |
| w/ BHQ) | | | | |
| HPV51 | TATGCGTGACCAGCTACCAG | 21 | 77 | E7 |
| forward | | | | |
| primer | | | | |
| HPV51 | AACACCTGCAACACGGAGCT | 22 | | |
| reverse | | | | |
| primer | | | | |
| HPV51 | CGTAGCCTGTCCAGCCCGTC | 39 | | |
| probe | | | | |
| (HEX- | | | | |
| labeled | | | | |
| w/ BHQ) | | | | |

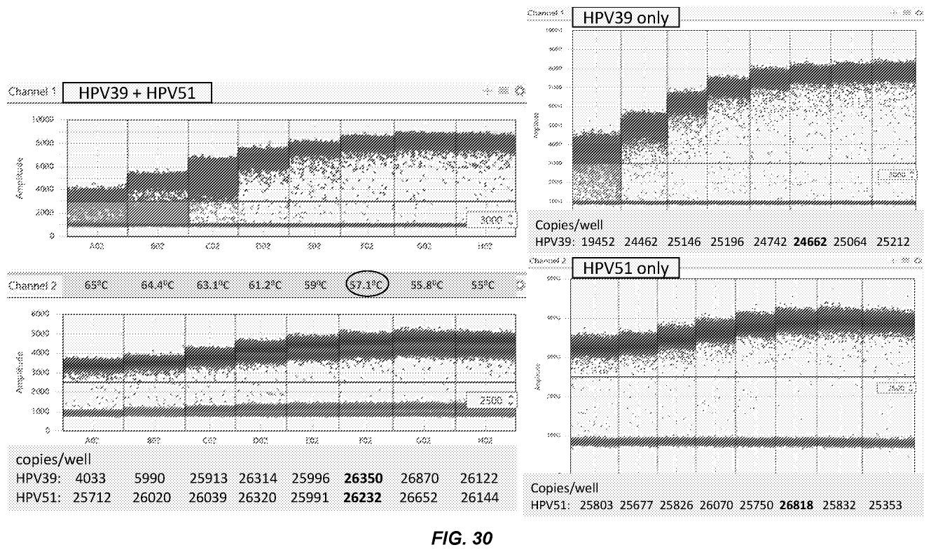

Amplification and detection with HPV39 and HPV51 primers and probes was tested at various annealing/extension temperatures from 55° C.-65° C., (FIG. 30) using nucleotide fragments comprising the amplicon sequences. 57.1° C. was identified as the best temperature for detection of both targets.

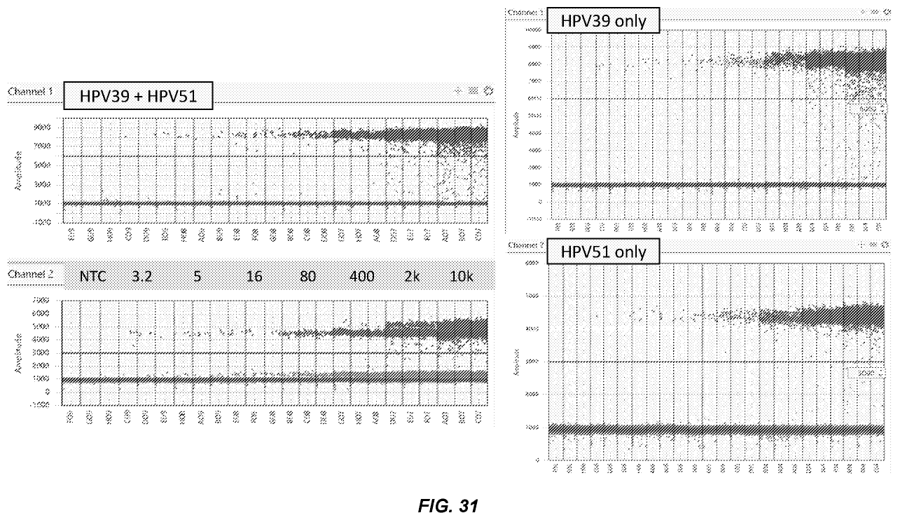

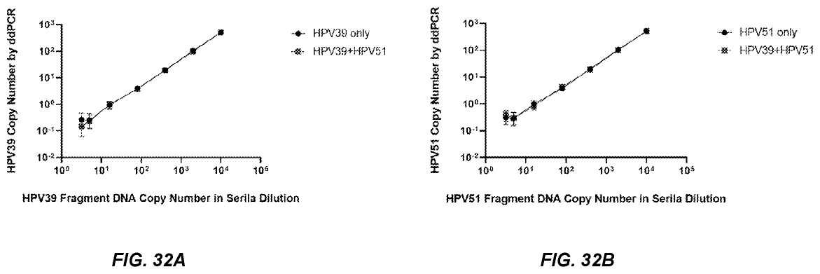

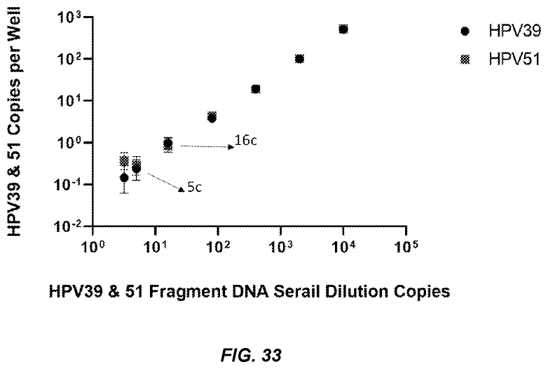

Amplification and detection with HPV39 and HPV51 primers and probes was tested using HPV39 and HPV51 fragments diluted from 10,000 (10 k) copies down to 3.2 copies (FIGS. 31-33). The limit of detection (LoD) was determined to be 5 copies of HPV39 DNA and 5 copies of HPV51 DNA per reaction.

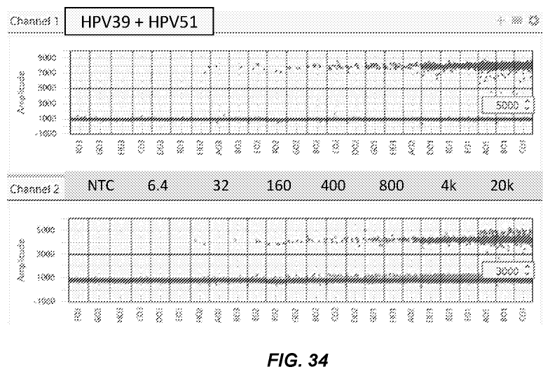

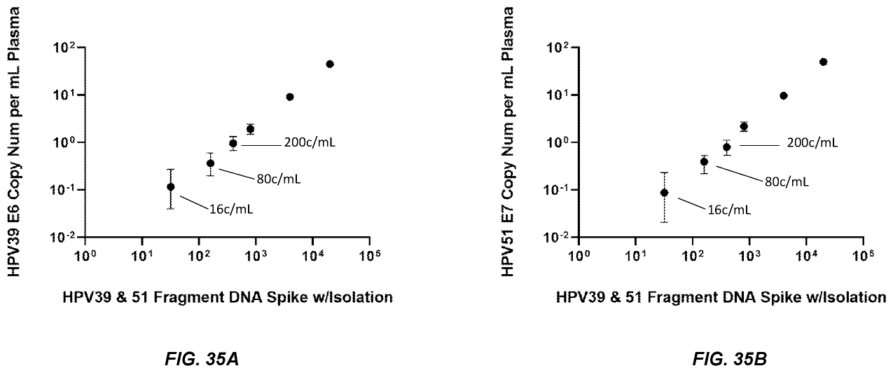

Amplification and detection with HPV39 and HPV51 primers and probes was tested using with HPV39 and HPV51 fragments diluted from 20,000 (20 k) copies down to 6.4 copies in plasma from non-HPV infected individuals. DNA was extracted from the spiked plasma samples and analyzed using the with HPV39 and HPV51 primers and probes via droplet digital PCR (FIGS. 34-35B). The limit of quantification (LoQ) in plasma was determined to be 16 copies per ml of plasma for both HPV52 and HPV59.

### Example 7—Analysis of Cross-Reactivity Between Different HPV Types

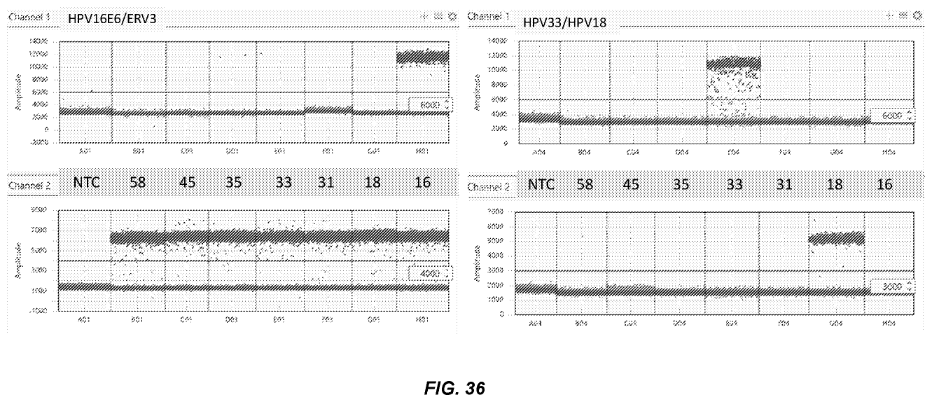

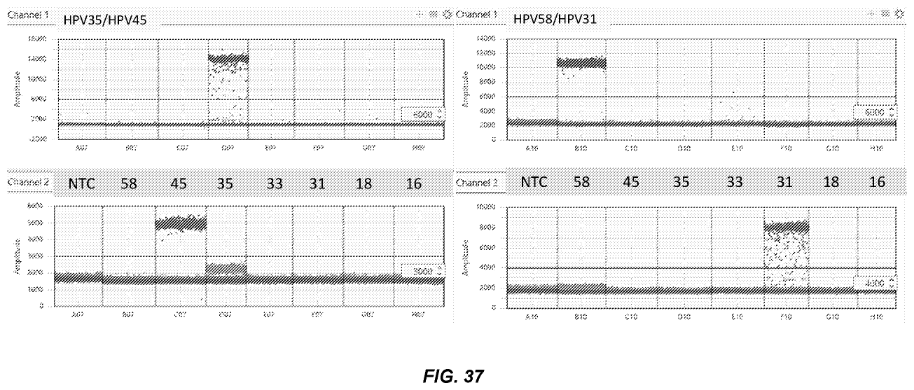

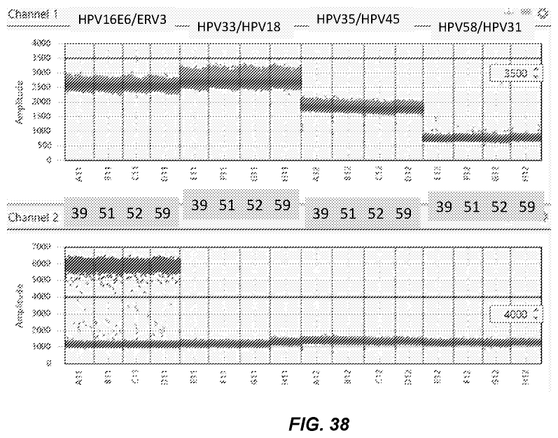

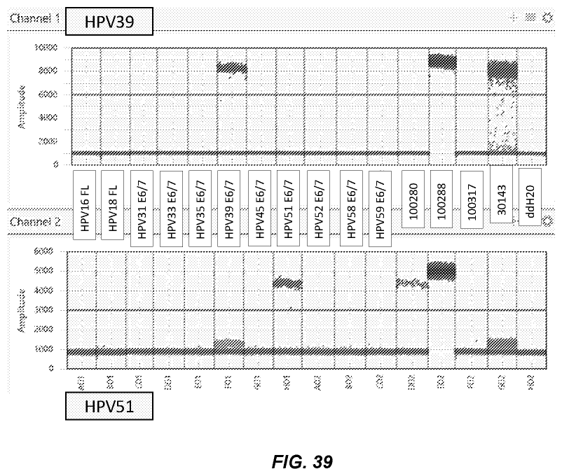

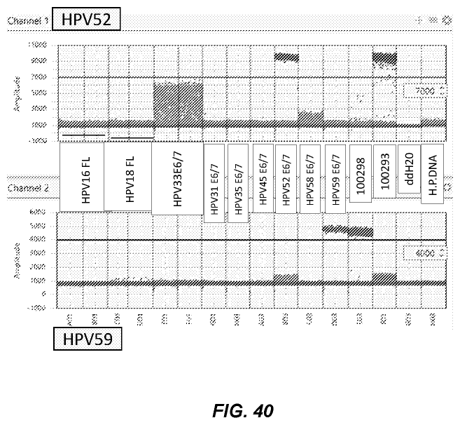

11 sets of plasmid DNA, each comprising one of full length HPV16, full length HPV18, HPV31 E6+E7, HPV33 E6+E7, HPV35 E6+E7, HPV39 E6+E7, HPV45 E6+E7, HPV51 E6+E7, HPV58 E6+E7, and HPV59 E6+E7, were amplified and detected using droplet digital PCR with the following primer and probe sets: HPV16 (FAM)+ERV3 (HEX); HPV33 (FAM)+HPV18 (HEX); HPV35 (FAM)+HPV45 (HEX); HPV58 (FAM)+HPV31 (HEX); HPV39 (FAM)+HPV51 (HEX); and HPV52 (FAM)+HPV59 (HEX). Cross-reactivity (i.e., off-target amplification) between each set was analyzed. The results are shown in Table 11 and Table 12 and FIGS. 36-40. No cross-reactivity was observed between any of the 11 HPV types.

TABLE 11

| | HPV16 | HPV18 | HPV31 | HPV33 | HPV35 | HPV39 | |
|---|---|---|---|---|---|---|---|
| | FL | FL | E6 + E7 | E6 + E7 | E6 + E7 | E6 + E7 | |
| ddPCR | (FAM/ | (FAM/ | (FAM/ | (FAM/ | (FAM/ | (FAM/ | |
| (copy/well) | HEX) | HEX) | HEX) | HEX) | HEX) | HEX) | Threshold |
| HPV16 (FAM) + | 2303/3910 | 0/11674 | 0/8690 | 1.73/9300 | 3.55/8786 | 0/0 | 6000, |
| ERV3 (HEX) | | | | | | | 4000 |
| HPV33 (FAM) + | 0/0 | 0/1260 | 0/0 | 1415/0 | 0/0 | 0/0 | 6000, |
| HPV18 (HEX) | | | | | | | 4000 |
| HPV35 (FAM) + | 0/0 | 0/0 | 0/0 | 0/0 | 1422/0 | 0/0 | 6000, |
| HPV45 (HEX) | | | | | | | 4000 |
| HPV58 (FAM) + | 0/0 | 0/0 | 0/1169 | 0/0 | 0/0 | 0/0 | 8000, |
| HPV31 (HEX) | | | | | | | 4000 |
| HPV39 (FAM) + | 0/0 | 0/0 | 0/0 | 0/0 | 0/0 | 447/0 | 5000, |
| HPV51 (HEX) | | | | | | | 3000 |
| HPV52 (FAM) + | 0/0 | 0/0 | 0/0 | 0/0 | 0/0 | 0/0 | 7000, |
| HPV59 (HEX) | | | | | | | 3000 |

TABLE 12

| | HPV45 | HPV51 | HPV52 | HPV58 | HPV59 | | |
|---|---|---|---|---|---|---|---|
| | E6 + E7 | E6 + E7 | FL | E6 + E7 | E6 + E7 | No | |
| ddPCR | (FAM/ | (FAM/ | (FAM/ | (FAM/ | (FAM/ | template | |
| (copy/well) | HEX) | HEX) | HEX) | HEX) | HEX) | control | Threshold |
| HPV16 (FAM) + | 0/9155 | 0/0 | 0/0 | 0/9455 | 0/0 | 1.61/0 | 6000, |
| ERV3 (HEX) | | | | | | | 4000 |
| HPV33 (FAM) + | 0/0 | 0/0 | 0/0 | 0/1.53 | 0/0 | 0/0 | 6000, |
| HPV18 (HEX) | | | | | | | 4000 |
| HPV35 (FAM) + | 0/1594 | 0/0 | 0/0 | 0/0 | 0/0 | 0/0 | 6000, |
| HPV45 (HEX) | | | | | | | 4000 |
| HPV58 (FAM) + | 0/0 | 0/0 | 0/0 | 1860/0 | 0/0 | 0/0 | 8000, |
| HPV31 (HEX) | | | | | | | 4000 |
| HPV39 (FAM) + | 0/0 | 0/329 | 0/0 | 0/0 | 0/0 | 0/1 | 5000, |
| HPV51 (HEX) | | | | | | | 3000 |
| HPV52 (FAM) + | 0/0 | 0/0 | 965/0 | 0/0 | 0/272 | 0/2 | 7000, |
| HPV59 (HEX) | | | | | | | 3000 |

### Example 8—Analysis of Metastatic HPV+ Relapse Using a Droplet Digital PCR HPV Assay

1 mL of plasma and 1 mL of serum were obtained from 16 patients having relapse of metastatic HPV+ carcinoma. Cell-free DNA (cfDNA) was extracted from each plasma sample and analyzed using a droplet digital PCR (ddPCR) assay. HPV16 forward primer (SEQ ID NO:1), HPV16 reverse primer (SEQ ID NO:2), HPV16 probe (SEQ ID NO:29), ERV-3 forward primer (SEQ ID NO:27), ERV-3 reverse primer (SEQ ID NO:28), and ERV-3 probe (SEQ ID NO:42) were used, following a procedure as described in Example 9. The results are shown in Table 13.

TABLE 13

| | HPV16 E6 (copies/1 mL | | ERV3 (copies/1 mL | |
|---|---|---|---|---|
| | sample) | | Sample) | |
| SampleID | Plasma | Serum | Plasma | Serum |
| 008 | 1061.54 | 996.92 | 6618.46 | 107538.46 |
| 009 | 5981.54 | 5243.08 | 4975.38 | 15415.38 |
| 015 | 132.92 | 121.85 | 987.69 | 28523.08 |
| 017 | 49200.00 | 55200.00 | 9932.31 | 158953.85 |
| 020 | 100.62 | 151.38 | 4652.31 | 117230.77 |
| 022 | 2473.85 | 2889.23 | 1227.69 | 45969.23 |
| 024 | 1209.23 | 1975.38 | 4846.15 | 60000.00 |
| 028 | 697.85 | 684.92 | 2261.54 | 31015.38 |
| 029 | 233353.85 | 254215.38 | 98861.54 | 362953.85 |
| 030 | 3646.15 | 3563.08 | 3396.92 | 34892.31 |
| 037 | 828.92 | 960.00 | 4680.00 | 129969.23 |
| 040 | 24646.15 | 25753.85 | 2584.62 | 284307.69 |
| 044 | 10338.46 | 11261.54 | 2547.69 | 142615.38 |
| 045 | 238800.00 | 263446.15 | 4587.69 | 470584.62 |
| 047 | 92861.54 | 97200.00 | 215907.69 | 382892.31 |
| 048 | 0.00 | 9.6 | 2464.62 | 35538.46 |
| ddH20 | | 0 | | 0 |
| HPV16/ERV3 | | 104.6 | | 15100 |
| positive control | | | | |
| (100 copies | | | | |
| HPV) | | | | |

### Example 9—Example Droplet Digital PCR HPV Assay

An assay is performed using the below equipment, materials, and procedure to detect HPV DNA from a biological sample.

### Equipment

1. QX200 ddPCR System (Reader & Generator)
2. C1000 Touch thermal cycler 1851197
3. PX1 PCR Plate Sealer 181-4000
4. Biosafety Cabinet
5. Rainin Single Channel Pipettes (p20, 200, 1000)
6. Rainin Multi-Channel Pipettes (LTS 2, 20, 200)
7. Vortex
8. Microcentrifuge

### Reagents/Materials

1. ddPCR™ x Supermix for Probes (No dUTP)
2. DG8 Cartridges for QX200-QX100 Droplet Generator
3. DG8 Gaskets for QX200-QX100 Droplet Generator
4. Droplet Generation Oil for Probes
5. ddPCR Plates 96-Well, Semi-Skirted
6. ddPCR™ Droplet Reader Oil 1863004 Bio-Rad
7. PCR Plate Heat Seal, foil, pieceable 1814040 Bio-Rad
8. 200-1000 μL Aerosol Barrier Pipette Tips
9. 20-200 μL Aerosol Barrier Pipette Tips
10. 0.2-20 μL Aerosol Barrier Pipette Tips
11. UltraPure Distilled Water
12. Custom designed primers & probes
13. Custom designed long oligonucleotide DNA for each type of HPV as positive control

### Procedure

1. Clean the surface of BSC and pipettes with 10% bleach and 70% ethanol.
2. Prepare ddPCR master mix for each set of targets as outlined in Table 14:

TABLE 14

| Reagents | 1 reaction (μL) | Final Con. | |
|---|---|---|---|
| 4x ddPCR Supermix for probe no | 5 | 1x | |
| dUTP | | | |
| Target 1 Primer F (100 uM) | 0.18 | 900 | nM |
| Target 1 Primer R (100 uM) | 0.18 | 900 | nM |
| Target 1 Probe-FAM (20 uM) | 0.25 | 250 | nM |
| BamHI (20 u/uL) | 0.2 | 4 | u/reaction |
| Target 2 Primer F (100 uM) | 0.18 | 900 | nM |
| Target 2 Primer R (100 uM) | 0.18 | 900 | nM |
| Target 2 Probe-HEX (20 uM) | 0.25 | 250 | nM |

| | |
|---|---|
| Template DNA (1-100,000 copies) | 13 |
| dH2O | 0.58 |
| Total Volume | 20 |

Note:
Positive and negative samples are to be include in each run. 5% extra volume should be used. All steps should be performed at room temperature.

3. Droplet generation:
   a) First load 20 μL of the reaction mixture into the sample well in the cartridge.
   b) Next, load 70 μL of droplet generation oil for probes into the oil wells.
   c) Attach a DG cartridge gasket.
   d) Place the assembled droplet cartridge into the QX200 droplet generator.
   e) Transfer 40 μL of the droplet/oil mixture into a 96-well plate slowly and carefully.
   f) Place a pierceable foil seal on the 96-well plate and seal the plate on the plate sealer at 180° C. for 5 seconds.
4. Program the C1000 Thermal Cycler using the cycler protocol outlined below in Table 15:

TABLE 15

| | Temperature | | | Ramp | Number |
|---|---|---|---|---|---|
| Cycling Step | (° C.) | Time | | Rate | of Cycles |
| Enzyme activation | 95 | 10 | min | 2° C./sec | 1 |
| Denaturation | 94 | 30 | sec | | 40 |
| Annealing/extension | 60 | 1 | min | | 40 |
| Enzyme deactivation | 98 | 10 | min | | 1 |

| | | | |
|---|---|---|---|
| Hold | 4 | infinite | 1 |

5. Configure the plate template under Quantasoft software.
6. Data analysis using Quantasoft Analysis Pro software to determine HPV types and copy number per well

### Example 10—Analysis of Various HPV Types in Combination with ERV3 HPV18

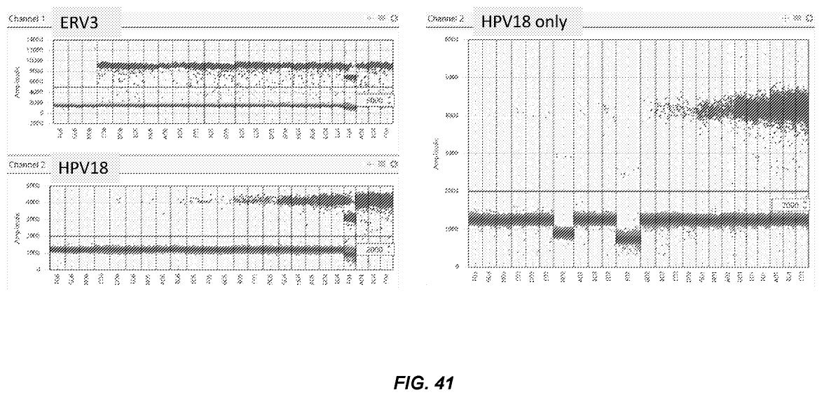

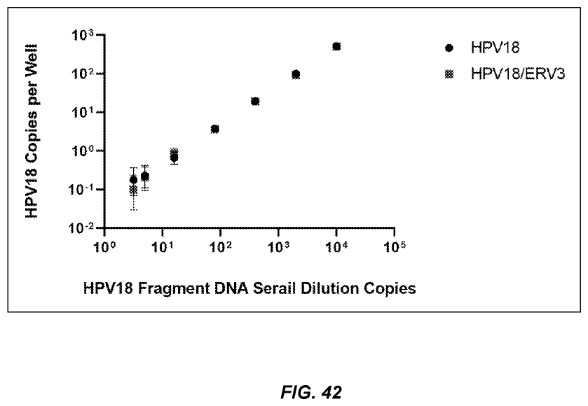

Amplification and detection with HPV18 and ERV3 primers and probes was tested using HPV18 and ERV3 fragments diluted from 10,000 (10 k) copies down to 3.2 copies run at Tm 57.1° C., (FIGS. 41-42 and Tables 16 and 17). HPV18 (HEX) and ERV3 (FAM) probes were used. 5 copies per reaction was the HPV18 sensitivity.

TABLE 16

| | HPV18 only | HPV18 + ERV3 |
|---|---|---|
| Loading Copies | HPV18 Copies/Well | HPV18 Copies/Well |
| 10000 | 10297.0 | 10215.0 |
| 2000 | 2001.0 | 1840.0 |
| 400 | 388.0 | 384.3 |
| 80 | 76.6 | 73.0 |
| 16 | 14.4 | 17.4 |
| 5 | 4.6 | 4.0 |
| 3.2 | 3.6 | 1.9 |
| 0 | 0.0 | 0.0 |

TABLE 17

| Loading | HPV18 + ERV3 | | |
|---|---|---|---|
| Copies | HPV18 Conc(copies/μL) | PoissonConfMax | PoissonConfMin |
| 10000 | 511 | 519 | 502 |
| 2000 | 92.2 | 95.7 | 88.8 |
| 400 | 19.2 | 20.6 | 17.7 |
| 80 | 3.64 | 4.24 | 3.04 |
| 16 | 0.866 | 1.19 | 0.609 |
| 5 | 0.206 | 0.385 | 0.0932 |
| 3.2 | 0.0989 | 0.233 | 0.0298 |
| 0 | 0 | 0.0789 | 0 |

### HPV31

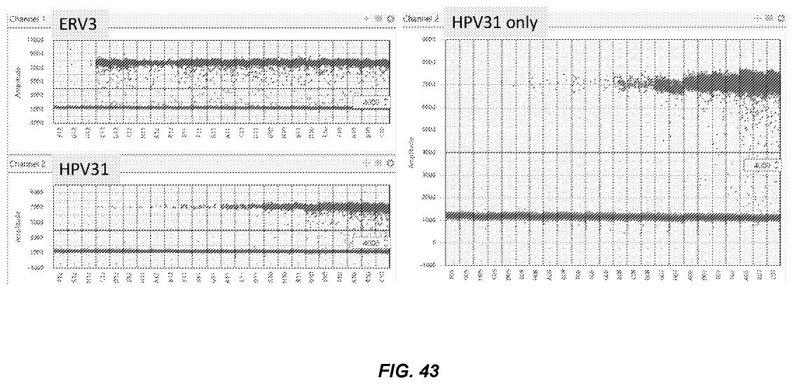

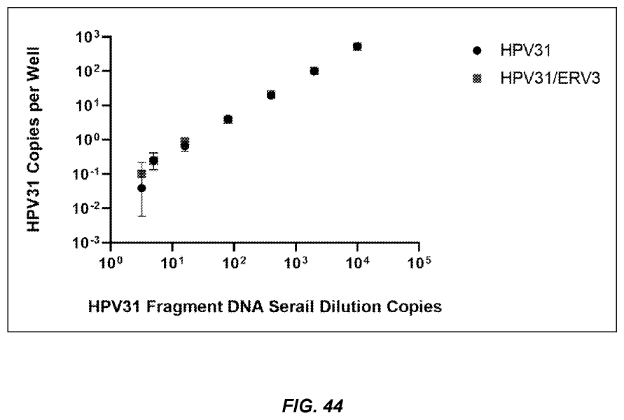

Amplification and detection with HPV31 and ERV3 primers and probes was tested using HPV31 and ERV3 fragments diluted from 10,000 (10 k) copies down to 3.2 copies run at Tm 57.1° C. (FIGS. 43-44 and Tables 18 and 19). HPV31 (HEX) and ERV3 (FAM) probes were used. 5 copies per reaction was the HPV31 sensitivity.

TABLE 18

| | HPV31 only | HPV31 + ERV3 |
|---|---|---|
| Loading Copies | HPV31 Copies/Well | HPV31 Copies/Well |
| 10000 | 10550.3 | 10312.0 |
| 2000 | 1980.3 | 2012.7 |
| 400 | 395.3 | 413.7 |
| 80 | 81.7 | 75.6 |
| 16 | 13.0 | 17.3 |
| 5 | 5.0 | 4.8 |
| 3.2 | 0.8 | 0.1 |
| 0 | 0.0 | 0.0 |

TABLE 19

| Loading | HPV31 + ERV3 | | |
|---|---|---|---|
| Copies | HPV31 Conc(copies/μL) | PoissonConfMax | PoissonConfMin |
| 10000 | 516 | 523 | 509 |
| 2000 | 101 | 103 | 97.8 |
| 400 | 20.7 | 21.9 | 19.4 |
| 80 | 3.79 | 4.32 | 3.26 |
| 16 | 0.868 | 1.15 | 0.638 |
| 5 | 0.243 | 0.408 | 0.129 |
| 3.2 | 0.101 | 0.22 | 0.0356 |
| 0 | 0 | 0.0584 | 0 |

### HPV33

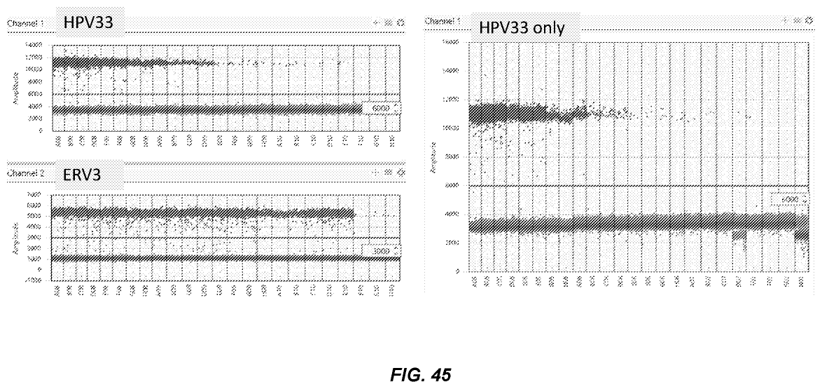

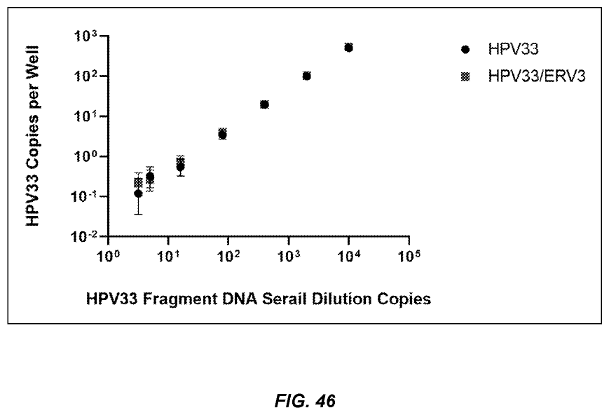

Amplification and detection with HPV33 and ERV3 primers and probes was tested using HPV33 and ERV3 fragments diluted from 10,000 (10 k) copies down to 3.2 copies run at Tm 57.1° C. (FIGS. 45-46 and Tables 20 and 21). HPV33 (FAM) and ERV3 (HEX) probes were used. 5 copies per reaction was the HPV33 sensitivity.

TABLE 20

| | HPV33 only | HPV33 + ERV3 |
|---|---|---|
| Loading Copies | HPV33 Conc(copies/μL) | HPV33 Conc(copies/μL) |
| 10000 | 10017.3 | 10756.0 |
| 2000 | 1987.0 | 2059.3 |
| 400 | 387.7 | 393.0 |
| 80 | 74.9 | 81.7 |
| 16 | 10.9 | 14.5 |
| 5 | 6.5 | 5.3 |
| 3.2 | 2.5 | 4.4 |
| 0 | 0.0 | 0.0 |

TABLE 21

| Loading | HPV33 + ERV3 | | |
|---|---|---|---|
| Copies | HPV33 Conc(copies/μL) | PoissonConfMax | PoissonConfMin |
| 10000 | 537 | 546 | 528 |
| 2000 | 103 | 106 | 99.5 |
| 400 | 19.7 | 21.2 | 18.2 |
| 80 | 4.09 | 4.72 | 3.46 |
| 16 | 0.725 | 1.03 | 0.488 |
| 5 | 0.265 | 0.456 | 0.137 |
| 3.2 | 0.215 | 0.39 | 0.103 |
| 0 | 0 | 0.0746 | 0 |

### HPV35

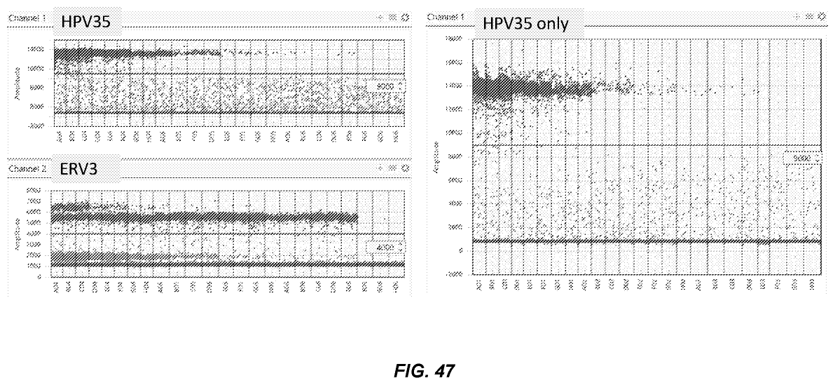

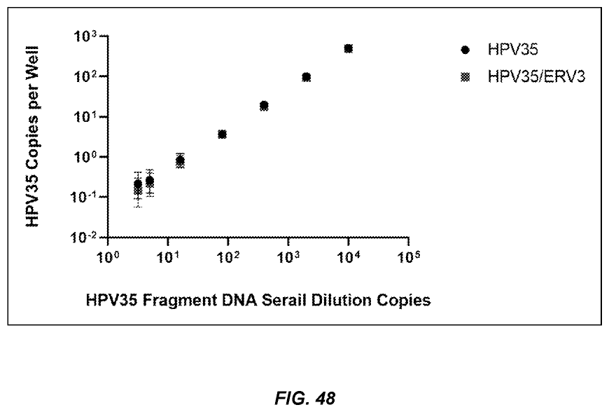

Amplification and detection with HPV35 and ERV3 primers and probes was tested using HPV35 and ERV3 fragments diluted from 10,000 (10 k) copies down to 3.2 copies run at Tm 57.1° C. (FIGS. 47-48 and Tables 22 and 23). HPV35 (FAM) and ERV3 (HEX) probes were used. 5 copies per reaction was the HPV35 sensitivity.

TABLE 22

| | HPV35 only | HPV35 + ERV3 |
|---|---|---|
| Loading Copies | HPV35 Conc(copies/μL) | HPV35 Conc(copies/μL) |
| 10000 | 10137.0 | 9834.0 |
| 2000 | 1996.7 | 1887.3 |
| 400 | 393.7 | 357.3 |
| 80 | 74.3 | 73.4 |
| 16 | 17.0 | 15.5 |
| 5 | 5.2 | 4.4 |
| 3.2 | 4.4 | 2.9 |
| 0 | 0.0 | 0.0 |

TABLE 23

| Loading | HPV35 + ERV3 | | |
|---|---|---|---|
| Copies | HPV35 Conc(copies/μL) | PoissonConfMax | PoissonConfMin |
| 10000 | 492 | 500 | 484 |
| 2000 | 94.4 | 97.7 | 91.1 |
| 400 | 17.8 | 19.2 | 16.5 |
| 80 | 3.68 | 4.25 | 3.12 |
| 16 | 0.775 | 1.08 | 0.536 |
| 5 | 0.218 | 0.395 | 0.104 |
| 3.2 | 0.146 | 0.298 | 0.0571 |
| 0 | 0 | 0.071 | 0 |

### HPV39

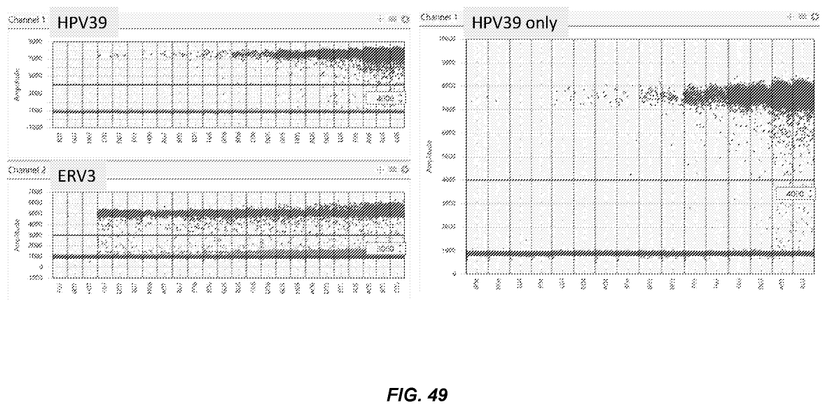

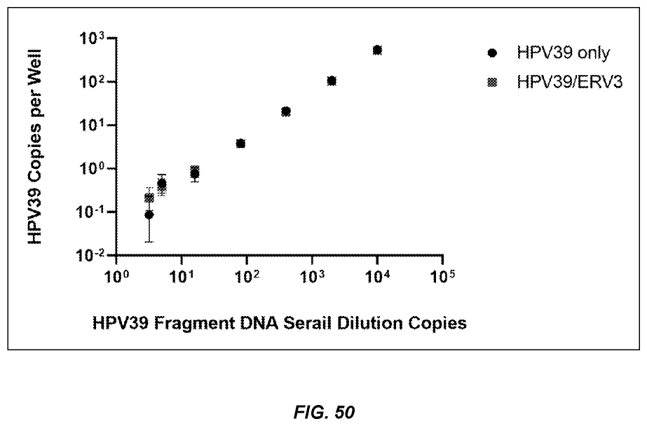

Amplification and detection with HPV39 and ERV3 primers and probes was tested using HPV39 and ERV3 fragments diluted from 10,000 (10 k) copies down to 3.2 copies run at Tm 57.1° C., (FIGS. 49-50 and Tables 24 and 25). HPV39 (FAM) and ERV3 (HEX) probes were used. 5 copies per reaction was the HPV39 sensitivity.

TABLE 24

| | HPV39 only | HPV39 + ERV3 |
|---|---|---|
| Loading Copies | HPV39 Copies/Well | HPV39 Copies/Well |
| 10000 | 10947.5 | 10508.0 |
| 2000 | 2147.0 | 2062.0 |
| 400 | 423.5 | 402.3 |
| 80 | 77.2 | 75.3 |
| 16 | 14.9 | 17.4 |
| 5 | 9.3 | 7.5 |
| 3.2 | 1.7 | 4.2 |
| 0 | 0.0 | 0.0 |

TABLE 25

| Loading | HPV39 + ERV3 | | |
|---|---|---|---|
| Copies | HPV39 Conc(copies/μL) | PoissonConfMax | PoissonConfMin |
| 10000 | 525 | 533 | 518 |
| 2000 | 103 | 106 | 100 |
| 400 | 20.1 | 21.4 | 18.9 |
| 80 | 3.76 | 4.29 | 3.23 |
| 16 | 0.866 | 1.15 | 0.634 |
| 5 | 0.38 | 0.573 | 0.236 |
| 3.2 | 0.21 | 0.362 | 0.109 |
| 0 | 0 | 0.071 | 0 |

### HPV45

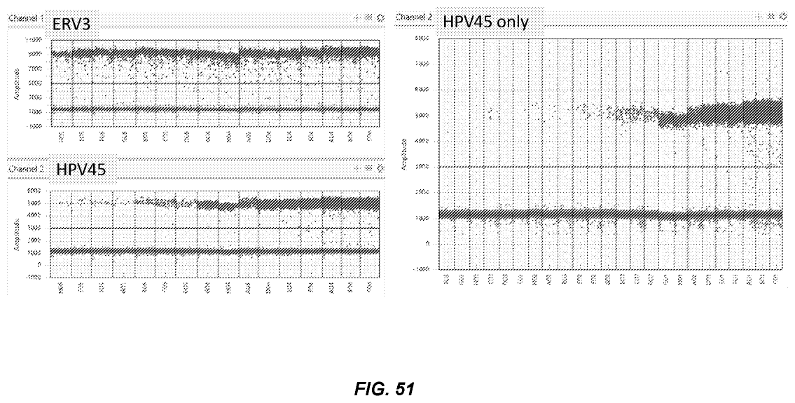

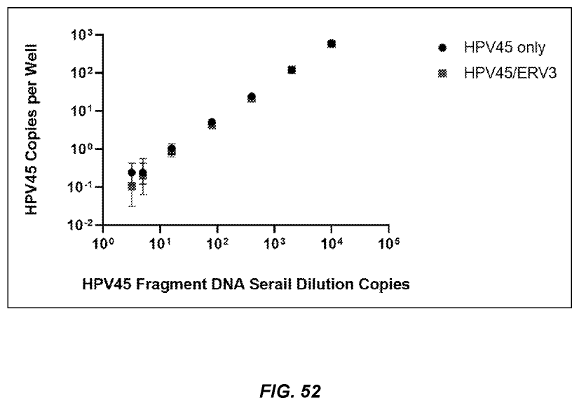

Amplification and detection with HPV45 and ERV3 primers and probes was tested using HPV45 and ERV3 fragments diluted from 10,000 (10 k) copies down to 3.2 copies run at Tm 57.1° C., (FIGS. 51-52 and Tables 26 and 27). HPV45 (HEX) and ERV3 (FAM) probes were used. 5 copies per reaction was the HPV45 sensitivity.

TABLE 26

| | HPV45 only | HPV45 + ERV3 |
|---|---|---|
| Loading Copies | HPV45 Copies/Well | HPV45 Copies/Well |
| 10000 | 11987.3 | 11555.3 |
| 2000 | 2417.3 | 2395.3 |
| 400 | 485.3 | 424.0 |
| 80 | 102.4 | 84.1 |
| 16 | 20.6 | 17.4 |
| 5 | 4.9 | 4.0 |
| 3.2 | 4.7 | 2.1 |
| 0 | 0.5 | 0.0 |

TABLE 27

| Loading | HPV45 + ERV3 | | |
|---|---|---|---|
| Copies | HPV45 Conc(copies/μL) | PoissonConfMax | PoissonConfMin |
| 10000 | 578 | 586 | 569 |
| 2000 | 120 | 123 | 116 |
| 400 | 21.2 | 22.6 | 19.8 |
| 80 | 4.2 | 4.82 | 3.58 |
| 16 | 0.871 | 1.18 | 0.619 |
| 5 | 0.199 | 0.56 | 0.0638 |
| 3.2 | 0.105 | 0.247 | 0.0316 |
| 0 | 0 | 0.0758 | 0 |

### HPV51

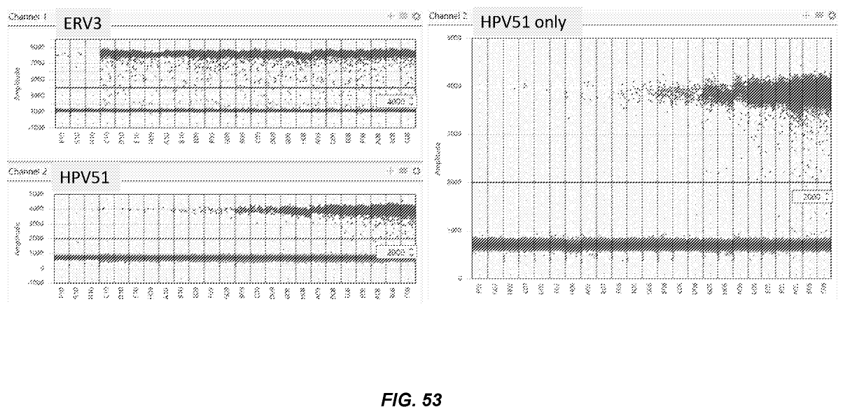

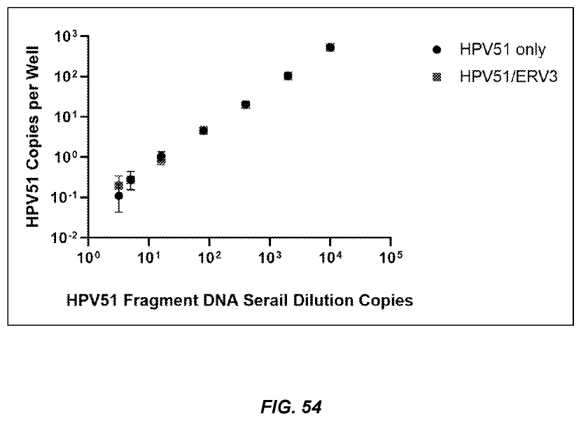

Amplification and detection with HPV51 and ERV3 primers and probes was tested using HPV51 and ERV3 fragments diluted from 10,000 (10 k) copies down to 3.2 copies run at Tm 57.1° C. (FIGS. 53-54 and Tables 28 and 29). HPV51 (HEX) and ERV3 (FAM) probes were used. 5 copies per reaction was the HPV51 sensitivity.

TABLE 28

| | HPV51 only | HPV51 + ERV3 |
|---|---|---|
| Loading Copies | HPV51 Copies/Well | HPV51 Copies/Well |
| 10000 | 10555.0 | 10535.0 |
| 2000 | 2084.0 | 2058.0 |
| 400 | 409.3 | 401.7 |
| 80 | 90.2 | 92.2 |
| 16 | 21.1 | 17.7 |
| 5 | 5.6 | 5.5 |
| 3.2 | 2.2 | 0.2 |
| 0 | 0.4 | 0.4 |

TABLE 29

| Loading | HPV51 + ERV3 | | |
|---|---|---|---|
| Copies | HPV51 Conc(copies/μL) | PoissonConfMax | PoissonConfMin |
| 10000 | 527 | 534 | 520 |
| 2000 | 103 | 106 | 100 |
| 400 | 20.1 | 21.3 | 18.9 |
| 80 | 4.61 | 5.19 | 4.02 |
| 16 | 0.888 | 1.17 | 0.657 |
| 5 | 0.273 | 0.442 | 0.153 |
| 3.2 | 0.195 | 0.344 | 0.0971 |
| 0 | 0.0195 | 0.093 | 0.000818 |

### HPV52

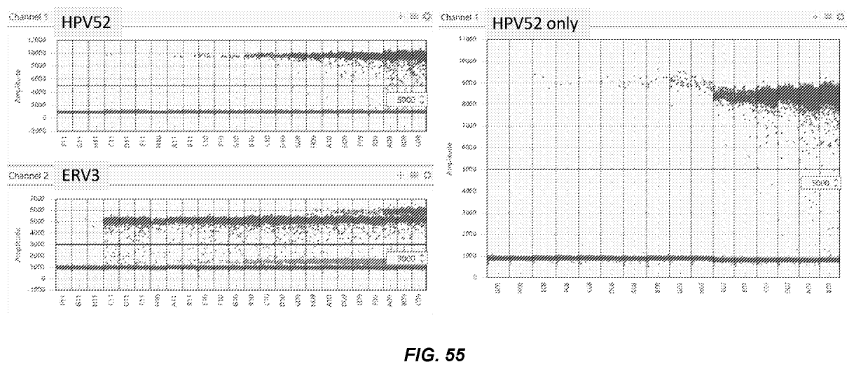

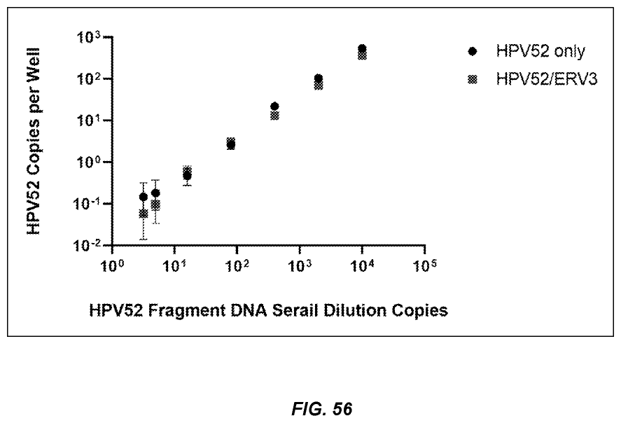

Amplification and detection with HPV52 and ERV3 primers and probes was tested using HPV52 and ERV3 fragments diluted from 10,000 (10 k) copies down to 3.2 copies run at Tm 57.1° C., (FIGS. 55-56 and Tables 30 and 31). HPV52 (FAM) and ERV3 (HEX) probes were used. 5 copies per reaction was the HPV52 sensitivity.

TABLE 30

| | HPV52 only | HPV52 + ERV3 |
|---|---|---|
| Loading Copies | HPV52 Copies/Well | HPV52 Copies/Well |
| 10000 | 7443.2 | 7376.0 |
| 2000 | 1425.2 | 1408.7 |
| 400 | 301.8 | 264.0 |
| 80 | 51.8 | 62.1 |
| 16 | 9.4 | 11.5 |
| 5 | 3.6 | 2.0 |
| 3.2 | 2.9 | 1.2 |
| 0 | 0.0 | 0.0 |

TABLE 31

| Loading | HPV52 + ERV3 | | |
|---|---|---|---|
| Copies | HPV52 Conc(copies/μL) | PoissonConfMax | PoissonConfMin |
| 10000 | 369 | 375 | 363 |
| 2000 | 70.5 | 72.8 | 68.1 |
| 400 | 13.2 | 14.2 | 12.2 |
| 80 | 3.11 | 3.59 | 2.62 |
| 16 | 0.578 | 0.816 | 0.392 |
| 5 | 0.097 | 0.211 | 0.0341 |
| 3.2 | 0.0589 | 0.156 | 0.014 |
| 0 | 0 | 0.0579 | 0 |

### HPV56

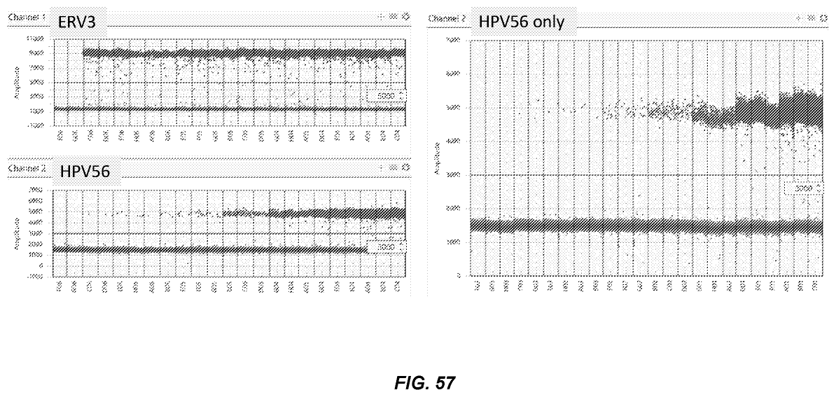

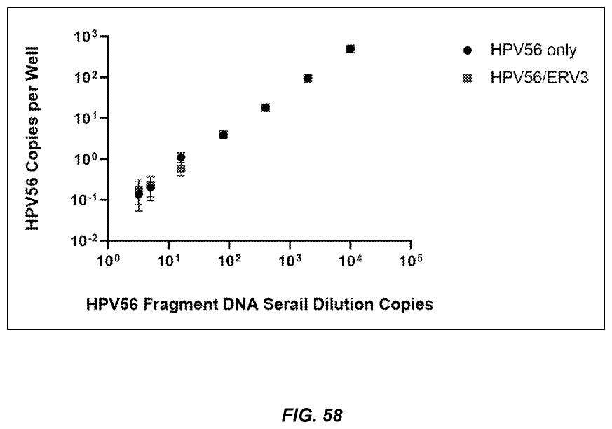

Amplification and detection with HPV56 and ERV3 primers and probes was tested using HPV56 and ERV3 fragments diluted from 10,000 (10 k) copies down to 3.2 copies run at Tm 57.1° C. (FIGS. 57-58 and Tables 32 and 33). HPV56 (HEX) and ERV3 (FAM) probes were used. 5 copies per reaction was the HPV56 sensitivity.

TABLE 32

| | HPV56 only | HPV56 + ERV3 |
|---|---|---|
| Loading Copies | HPV56 Copies/Well | HPV56 Copies/Well |
| 10000 | 10079.0 | 10106.7 |
| 2000 | 1949.7 | 1897.0 |
| 400 | 357.3 | 365.7 |
| 80 | 77.7 | 81.5 |
| 16 | 22.4 | 11.7 |
| 5 | 4.0 | 4.7 |
| 3.2 | 2.7 | 0.2 |
| 0 | 0.0 | 0.0 |

TABLE 33

| Loading | HPV56 + ERV3 | | |
|---|---|---|---|
| Copies | HPV56 Conc(copies/μL) | PoissonConfMax | PoissonConfMin |
| 10000 | 505 | 513 | 498 |
| 2000 | 94.9 | 97.7 | 92 |
| 400 | 18.3 | 19.6 | 17.1 |
| 80 | 4.05 | 4.63 | 3.48 |
| 16 | 0.584 | 0.829 | 0.393 |
| 5 | 0.23 | 0.386 | 0.119 |
| 3.2 | 0.171 | 0.32 | 0.0772 |
| 0 | 0 | 0.0994 | 0 |

### HPV58

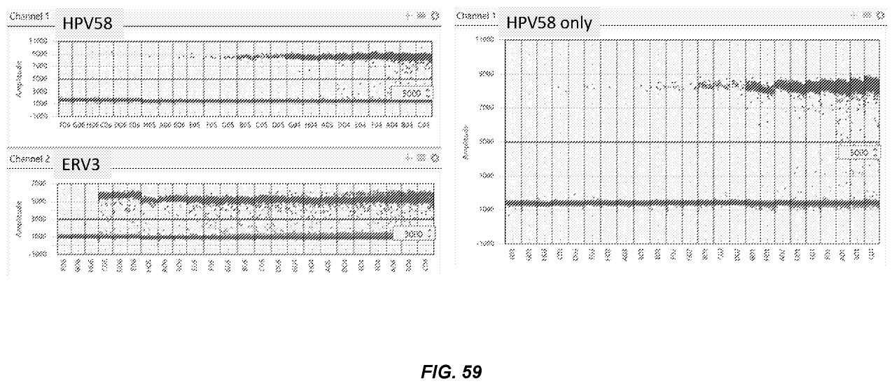

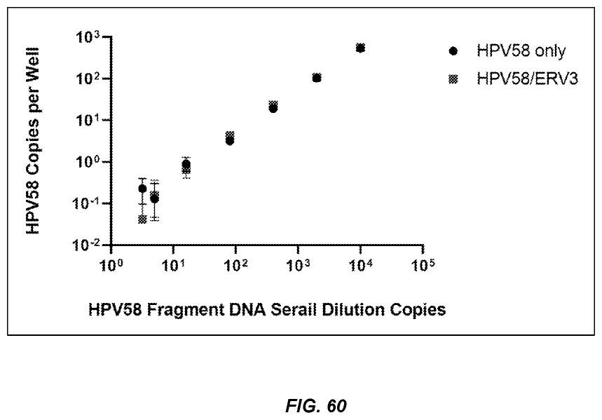

Amplification and detection with HPV58 and ERV3 primers and probes was tested using HPV58 and ERV3 fragments diluted from 10,000 (10 k) copies down to 3.2 copies run at Tm 57.1° C., (FIGS. 59-60 and Tables 34 and 35). HPV58 (FAM) and ERV3 (HEX) probes were used. 5 copies per reaction was the HPV58 sensitivity.

TABLE 34

| | HPV58 only | HPV58 + ERV3 |
|---|---|---|
| Loading Copies | HPV58 Copies/Well | HPV58 Copies/Well |
| 10000 | 10640.9 | 11289.7 |
| 2000 | 2059.8 | 2146.5 |
| 400 | 376.8 | 464.1 |
| 80 | 63.7 | 86.1 |
| 16 | 18.0 | 13.3 |
| 5 | 2.6 | 3.0 |
| 3.2 | 4.6 | 1.0 |
| 0 | 0.0 | 0.0 |

TABLE 35

| Loading | HPV58 + ERV3 | | |
|---|---|---|---|
| Copies | HPV58 Conc(copies/μL) | PoissonConfMax | PoissonConfMin |
| 10000 | 564 | 5745 | 554 |
| 2000 | 107 | 111 | 103 |
| 400 | 23.1 | 25 | 21.4 |
| 80 | 4.32 | 5.1 | 3.55 |
| 16 | 0.664 | 1.0115 | 0.408 |
| 5 | 0.154 | 0.364 | 0.0466 |
| 3.2 | 0.04165 | 0.1989 | 0.00175 |
| 0 | 0 | 0.0789 | 0 |

### HPV59

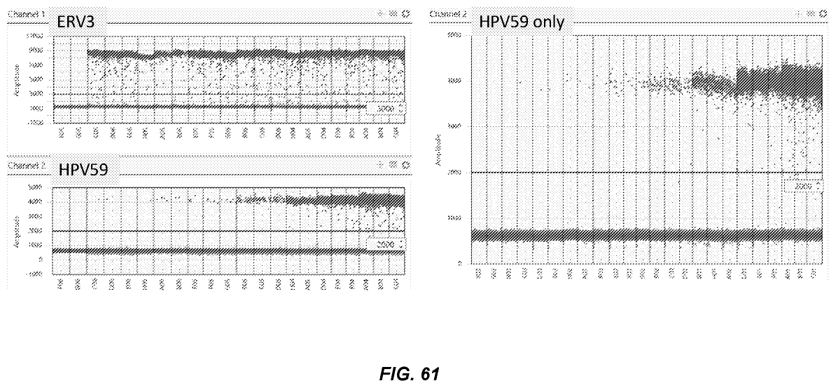

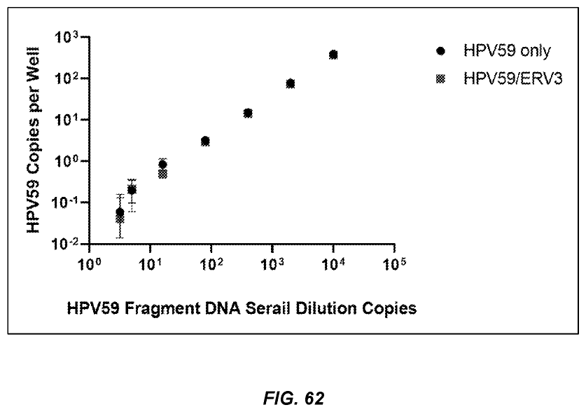

Amplification and detection with HPV59 and ERV3 primers and probes was tested using HPV59 and ERV3 fragments diluted from 10,000 (10 k) copies down to 3.2 copies run at Tm 57.1° C. (FIGS. 61-62 and Tables 36 and 37). HPV59 (HEX) and ERV3 (FAM) probes were used. 5 copies per reaction was the HPV59 sensitivity.

TABLE 36

| | HPV59 only | HPV59 + ERV3 |
|---|---|---|
| Loading Copies | HPV59 Copies/Well | HPV59 Copies/Well |
| 10000 | 7792.7 | 7366.3 |
| 2000 | 1559.7 | 1467.7 |
| 400 | 301.3 | 282.5 |
| 80 | 63.5 | 58.8 |
| 16 | 16.8 | 10.0 |
| 5 | 3.9 | 4.2 |
| 3.2 | 1.2 | 0.0 |
| 0 | 0.0 | 0.0 |

TABLE 37

| Loading | HPV59 + ERV3 | | |
|---|---|---|---|
| Copies | HPV59 Conc(copis/μL) | PoissonConfMax | PoissonConfMin |
| 10000 | 368 | 374 | 362 |
| 2000 | 73.3 | 76 | 70.6 |
| 400 | 14.2 | 15.5 | 12.8 |
| 80 | 2.93 | 3.42 | 2.45 |
| 16 | 0.499 | 0.724 | 0.387 |
| 5 | 0.21 | 0.365 | 0.0603 |
| 3.2 | 0.0409 | 0.131 | 0.0062 |
| 0 | 0 | 0.0903 | 0 |

### HPV68

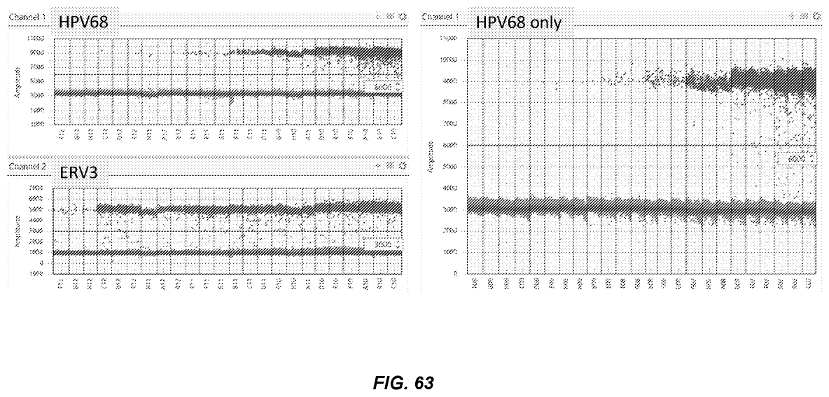

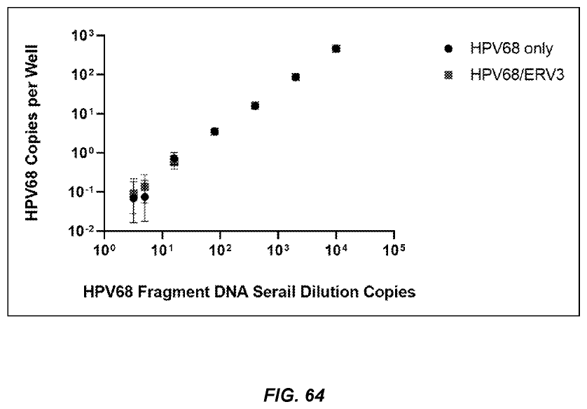

Amplification and detection with HPV68 and ERV3 primers and probes was tested using HPV68 and ERV3 fragments diluted from 10,000 (10 k) copies down to 3.2 copies run at Tm 57.1° C. (FIGS. 63-64 and Tables 38 and 39). HPV68 (FAM) and ERV3 (HEX) probes were used. 5 copies per reaction was the HPV68 sensitivity.

TABLE 38

| | HPV68 only | HPV68 + ERV3 |
|---|---|---|
| Loading Copies | HPV68 Conc(copies/μL) | HPV68 Conc(copies/μL) |
| 10000 | 9273.0 | 9240.0 |
| 2000 | 1727.7 | 1735.3 |
| 400 | 314.7 | 326.3 |
| 80 | 70.7 | 69.2 |
| 16 | 14.5 | 11.8 |
| 5 | 1.5 | 2.6 |
| 3.2 | 1.4 | 1.9 |
| 0 | 0.0 | 0.0 |

TABLE 39

| Loading | HPV68 + ERV3 | | |
|---|---|---|---|
| Copies | HPV68 Conc(copies/μL) | PoissonConfMax | PoissonConfMin |
| 10000 | 462 | 469 | 455 |
| 2000 | 86.9 | 89.7 | 84 |
| 400 | 16.3 | 17.5 | 15.1 |
| 80 | 3.49 | 4.05 | 2.92 |
| 16 | 0.582 | 0.842 | 0.382 |
| 5 | 0.134 | 0.274 | 0.0525 |
| 3.2 | 0.0918 | 0.216 | 0.0277 |
| 0 | 0 | 0.0683 | 0 |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed:

1. A method for detecting HPV DNA in a biological sample from a subject, the method comprising:
(a) amplifying HPV DNA from the biological sample using at least six primer pairs each configured to amplify a different HPV type of a set of high-risk HPV types; and
(b) detecting which HPV types of the set of high-risk HPV types are present in the biological sample;
wherein the set of high-risk HPV types comprises six or more of HPV16, HPV18, HPV31, HPV33, HPV35, HPV39, HPV45, HPV51, HPV52, HPV58, HPV59, HPV56, and HPV68.

2. The method of claim 1, wherein the at least six primer pairs are each configured to amplify a region of the HPV E6 gene or the HPV E7 gene.

3. The method of claim 1 or 2, wherein (b) comprises using at least six probes each configured to bind to a different HPV type of the set of high-risk HPV types.

4. The method of any of claims 1-3, wherein the set of high-risk HPV types comprises seven or more of HPV16, HPV18, HPV31, HPV33, HPV35, HPV39, HPV45, HPV51, HPV52, HPV58, HPV59, HPV56, and HPV68.

5. The method of any of claims 1-4, wherein the set of high-risk HPV types comprises eight or more of HPV16, HPV18, HPV31, HPV33, HPV35, HPV39, HPV45, HPV51, HPV52, HPV58, HPV59, HPV56, and HPV68.

6. The method of any of claims 1-5, wherein the set of high-risk HPV types comprises nine or more of HPV16, HPV18, HPV31, HPV33, HPV35, HPV39, HPV45, HPV51, HPV52, HPV58, HPV59, HPV56, and HPV68.

7. The method of any of claims 1-6, wherein the set of high-risk HPV types comprises ten or more of HPV16, HPV18, HPV31, HPV33, HPV35, HPV39, HPV45, HPV51, HPV52, HPV58, HPV59, HPV56, and HPV68.

8. The method of any of claims 1-7, wherein the set of high-risk HPV types comprises eleven or more of HPV16, HPV18, HPV31, HPV33, HPV35, HPV39, HPV45, HPV51, HPV52, HPV58, HPV59, HPV56, and HPV68.

9. The method of any of claims 1-8, wherein the set of high-risk HPV types comprises twelve or more of HPV16, HPV18, HPV31, HPV33, HPV35, HPV39, HPV45, HPV51, HPV52, HPV58, HPV59, HPV56, and HPV68.

10. The method of any of claims 1-9, wherein the set of high-risk HPV types comprises HPV16, HPV18, HPV31, HPV33, HPV35, HPV39, HPV45, HPV51, HPV52, HPV58, HPV59, HPV56, and HPV68.

11. The method of any of claims 1-10, wherein the at least six primer pairs comprise at least six of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs: 13 and 14, (viii) SEQ ID NOs: 15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26.

12. The method of any of claims 1-11, wherein the at least six primer pairs comprise at least seven primer pairs comprising at least seven of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26.

13. The method of any of claims 1-12, wherein the at least six primer pairs comprise at least eight primer pairs comprising at least eight of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26.

14. The method of any of claims 1-13, wherein the at least six primer pairs comprise at least nine primer pairs comprising at least nine of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26.

15. The method of any of claims 1-14, wherein the at least six primer pairs comprise at least ten primer pairs comprising at least ten of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26.

16. The method of any of claims 1-15, wherein the at least six primer pairs comprise at least eleven primer pairs comprising at least eleven of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26.

17. The method of any of claims 1-16, wherein the at least six primer pairs comprise at least twelve primer pairs comprising at least twelve of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs: 17 and 18, (x) SEQ ID NOs: 19 and 20, and (xi) SEQ ID NOs:21 and 22, (xii) SEQ ID NOs:23 and 25, and (xiii) SEQ ID NOs:24 and 26.

18. The method of any of claims 1-17, wherein the at least six primer pairs comprise at least thirteen primer pairs comprising (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs: 19 and 20, and (xi) SEQ ID NOs:21 and 22, (xii) SEQ ID NOs:23 and 25, and (xiii) SEQ ID NOs:24 and 26.

19. The method of any of claims 1-16, wherein a first primer pair of the at least six primer pairs is configured to amplify HPV16 DNA.

20. The method of claim 19 or 20, wherein the detecting of (b) comprises using a first probe comprising SEQ ID NO:29.

21. The method of any of claims 19-20, wherein the first primer pair comprises a first forward primer comprising SEQ ID NO:1 and a first reverse primer comprising SEQ ID NO:2.

22. The method of any of claims 1-21, wherein a second primer pair of the at least six primer pairs is configured to amplify HPV18 DNA.

23. The method of claim 22, wherein the detecting of (b) comprises using a second probe comprising SEQ ID NO:30.

24. The method of any of claims 22-23, wherein the second primer pair comprises a second forward primer comprising SEQ ID NO:3 and a second reverse primer comprising SEQ ID NO:4.

25. The method of any of claims 1-24, wherein a third primer pair of the at least six primer pairs is configured to amplify HPV33 DNA.

26. The method of claim 25, wherein the detecting of (b) comprises using a third probe comprising SEQ ID NO:31.

27. The method of any of claims 25-26, wherein the third primer pair comprises a third forward primer comprising SEQ ID NO:5 and a third reverse primer comprising SEQ ID NO:6.

28. The method of any of claims 1-27, wherein a fourth primer pair of the at least six primer pairs is configured to amplify HPV35 DNA.

29. The method of claim 28, wherein the detecting of (b) comprises using a fourth probe comprising SEQ ID NO:32.

30. The method of any of claims 28-29, wherein the fourth primer pair comprises a fourth forward primer comprising SEQ ID NO:7 and a fourth reverse primer comprising SEQ ID NO:8.

31. The method of any of claims 1-30, wherein a fifth primer pair of the at least six primer pairs is configured to amplify HPV45 DNA.

32. The method of claim 31, wherein the detecting of (b) comprises using a fifth probe comprising SEQ ID NO:33.

33. The method of any of claims 31-32, wherein the fifth primer pair comprises a fifth forward primer comprising SEQ ID NO:9 and a fifth reverse primer comprising SEQ ID NO:10.

34. The method of any of claims 1-33, wherein a sixth primer pair of the at least six primer pairs is configured to amplify HPV31 DNA.

35. The method of claim 34, wherein the detecting of (b) comprises using a sixth probe comprising SEQ ID NO:34.

36. The method of any of claims 34-35, wherein the sixth primer pair comprises a sixth forward primer comprising SEQ ID NO:11 and a sixth reverse primer comprising SEQ ID NO:12.

37. The method of any of claims 1-36, wherein the at least six primer pairs comprise at least seven primer pairs.

38. The method of claim 37, wherein a seventh primer pair of the at least seven primer pairs is configured to amplify HPV58 DNA.

39. The method of claim 38, wherein the detecting of (b) comprises using a seventh probe comprising SEQ ID NO:35.

40. The method of any of claims 38-40, wherein the seventh primer pair comprises a seventh forward primer comprising SEQ ID NO:13 and a seventh reverse primer comprising SEQ ID NO: 14.

41. The method of any of claims 1-40, wherein the at least six primer pairs comprise at least eight primer pairs.

42. The method of claim 41, wherein an eighth primer pair of the at least eight primer pairs is configured to amplify HPV52 DNA.

43. The method of claim 41 or 42, wherein the detecting of (b) comprises using an eighth probe comprising SEQ ID NO:36.

44. The method of any of claims 42-43, wherein the eighth primer pair comprises an eighth forward primer comprising SEQ ID NO:15 and an eighth reverse primer comprising SEQ ID NO:16.

45. The method of any of claims 1-44, wherein the at least six primer pairs are at least nine primer pairs.

46. The method of claim 45, wherein a ninth primer pair of the at least nine primer pairs is configured to amplify HPV59 DNA.

47. The method of claim 46, wherein the detecting of (b) comprises using a ninth probe comprising SEQ ID NO:37.

48. The method of claim 46 or 47, wherein the ninth primer pair comprises a ninth forward primer comprising SEQ ID NO: 17 and a ninth reverse primer comprising SEQ ID NO:18.

49. The method of any of claims 1-48, wherein the at least six primer pairs are at least ten primer pairs.

50. The method of claim 49, wherein a tenth primer pair of the at least ten primer pairs is configured to amplify HPV39 DNA.

51. The method of claim 50, wherein the detecting of (b) comprises using a tenth probe comprising SEQ ID NO:38.

52. The method of claim 50 or 51, wherein the tenth primer pair comprises a tenth forward primer comprising SEQ ID NO:19 and a tenth reverse primer comprising SEQ ID NO:20.

53. The method of any of claims 1-52, wherein the at least six primer pairs are at least eleven primer pairs.

54. The method of claim 53, wherein an eleventh primer pair of the at least eleven primer pairs is configured to amplify HPV51 DNA.

55. The method of claim 54, wherein the detecting of (b) comprises using an eleventh probe comprising SEQ ID NO:39.

56. The method of any of claims 54-55, wherein the eleventh primer pair comprises an eleventh forward primer comprising SEQ ID NO:21 and an eleventh reverse primer comprising SEQ ID NO:22.

57. The method of any of claims 1-56, wherein the at least six primer pairs are at least twelve primer pairs.

58. The method of claim 57, wherein a twelfth primer pair of the at least twelve primer pairs is configured to amplify HPV56 DNA.

59. The method of claim 58, wherein the detecting of (b) comprises using a twelfth probe comprising SEQ ID NO:40.

60. The method of any of claims 57-59, wherein the twelfth primer pair comprises a twelfth forward primer comprising SEQ ID NO:23 and a twelfth reverse primer comprising SEQ ID NO:25.

61. The method of any of claims 1-60, wherein the at least six primer pairs are at least thirteen primer pairs.

62. The method of claim 61, wherein a thirteenth primer pair of the at least thirteen primer pairs is configured to amplify HPV68 DNA.

63. The method of claim 62, wherein the detecting of (b) comprises using a thirteenth probe comprising SEQ ID NO:41.

64. The method of any of claims 61-63, wherein the thirteenth primer pair comprises a thirteenth forward primer comprising SEQ ID NO:23 and a thirteenth reverse primer comprising SEQ ID NO:25.

65. The method of any of claims 1-64, further comprising amplifying genomic DNA from the biological sample using an additional primer pair, wherein the additional primer pair is configured to amplify a region of Endogenous Retrovirus Group 3 (ERV-3).

66. The method of claim 65, wherein the additional primer pair amplifies a region of ERV-3 comprising SEQ ID:LLL.

67. The method of claim 65 or 66, wherein the detecting of (b) comprises using an additional probe comprising SEQ ID NO:42.

68. The method of any of claims 65-67, wherein the additional primer pair comprises an additional forward primer comprising SEQ ID NO:27 and an additional reverse primer comprising SEQ ID NO:28.

69. The method of any of claims 1-68, wherein the biological sample is a blood, plasma, or serum sample.

70. The method of any of claims 1-69, wherein the HPV DNA is from one or more tumor cells.

71. The method of any of claims 1-69, wherein the HPV DNA is cell-free DNA.

72. The method of any of claims 1-68, wherein the biological sample is a tissue sample.

73. The method of any of claims 1-72, wherein the biological sample contains less than 100 copies of HPV DNA.

74. The method of claim 73, wherein the biological sample contains less than 20 copies of HPV DNA.

75. The method of claim 74, wherein the biological sample contains less than 10 copies of HPV DNA.

76. The method of claim 75, wherein the biological sample contains less than 6 copies of HPV DNA.

77. The method of any of claims 1-76, further comprising, prior to (a), partitioning the HPV DNA into a plurality of partitions.

78. The method of claim 77, wherein the plurality of partitions is a plurality of droplets.

79. The method of claim 77 or 78, wherein (b) comprises detecting one or more signals from the plurality of partitions.

80. The method of any of claims 1-79, further comprising quantifying an amount of each of the HPV types in the biological sample.

81. The method of any of claims 1-80, further comprising diagnosing the subject with an HPV-associated cancer.

82. The method of claim 81, wherein the HPV-associated cancer is head and neck squamous cell carcinoma.

83. The method of claim 81 or 82, further comprising treating the subject for the HPV-associated cancer.

84. A method for detecting HPV DNA in a biological sample from a subject, the method comprising:
(a) amplifying HPV DNA from the biological sample using at least six primer pairs comprising at least six of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs: 15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26; and
(b) detecting the HPV DNA in the biological sample using at least six probes comprising at least six of SEQ ID NOs:29-41.

85. The method of claim 84, wherein the at least six primer pairs comprise at least seven primer pairs comprising at least seven of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26.

86. The method of claim 84 or 85, wherein the at least six primer pairs comprise at least eight primer pairs comprising at least eight of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26.

87. The method of any of claims 84-86, wherein the at least six primer pairs comprise at least nine primer pairs comprising at least nine of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26.

88. The method of any of claims 84-87, wherein the at least six primer pairs comprise at least ten primer pairs comprising at least ten of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26.

89. The method of any of claims 84-88, wherein the at least six primer pairs comprise at least eleven primer pairs comprising at least eleven of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26.

90. The method of any of claims 84-89, wherein the at least six primer pairs comprise at least twelve primer pairs comprising at least twelve of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs: 13 and 14, (viii) SEQ ID NOs:15 and 16, (viv) SEQ ID NOs: 17 and 18, (x) SEQ ID NOs: 19 and 20, and (xi) SEQ ID NOs:21 and 22, (xii) SEQ ID NOs:23 and 25, and (xiii) SEQ ID NOs:24 and 26.

91. The method of any of claims 84-90, wherein the at least six primer pairs are at least thirteen primer pairs comprising (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs: 15 and 16, (viv) SEQ ID NOs: 17 and 18, (x) SEQ ID NOs:19 and 20, and (xi) SEQ ID NOs:21 and 22, (xii) SEQ ID NOs:23 and 25, and (xiii) SEQ ID NOs:24 and 26.

92. The method of any of claims 84-89, further comprising amplifying genomic DNA from the biological sample using an additional primer pair comprising SEQ ID NOs:27 and 28.

93. The method of any of claims 84-92, wherein the at least six probes comprise at least seven probes comprising at least seven of SEQ ID NOs:29-41.

94. The method of any of claims 84-93, wherein the at least six probes comprise at least eight probes comprising at least eight of SEQ ID NOs:29-41.

95. The method of any of claims 84-94, wherein the at least six probes comprise at least nine probes comprising at least nine of SEQ ID NOs:29-41.

96. The method of any of claims 84-95, wherein the at least six probes comprise at least ten probes comprising at least ten of SEQ ID NOs:29-41.

97. The method of any of claims 84-96, wherein the at least six probes comprise at least eleven probes comprising at least eleven of SEQ ID NOs:29-41.

98. The method of any of claims 84-97, wherein the at least six probes comprise at least twelve probes comprising at least twelve of SEQ ID NOs:29-41.

99. The method of any of claims 84-98, wherein the at least six probes comprise at least thirteen probes comprising SEQ ID NOs:29-41.

100. The method of any of claims 84-97, further comprising detecting the gnomic DNA from the biological sample using an additional probe comprising SEQ ID NO:42.

101. The method of any of claims 84-100, wherein the biological sample is a blood, plasma, or serum sample.

102. The method of any of claims 84-101, wherein the HPV DNA is from one or more tumor cells.

103. The method of any of claims 84-101, wherein the HPV DNA is cell-free DNA.

104. The method of any of claims 84-100, wherein the biological sample is a tissue sample.

105. The method of any of claims 84-104, wherein the biological sample contains less than 100 copies of HPV DNA.

106. The method of claim 105, wherein the biological sample contains less than 20 copies of HPV DNA.

107. The method of claim 106, wherein the biological sample contains less than 10 copies of HPV DNA.

108. The method of claim 107, wherein the biological sample contains less than 6 copies of HPV DNA.

109. The method of any of claims 84-108, further comprising, prior to (a), partitioning the HPV DNA into a plurality of partitions.

110. The method of claim 109, wherein the plurality of partitions is a plurality of droplets.

111. The method of claim 109 or 110, wherein (b) comprises detecting one or more signals from the plurality of partitions.

112. The method of any of claims 84-111, further comprising quantifying an amount of each of the HPV types in the biological sample.

113. The method of any of claims 84-112, further comprising diagnosing the subject with an HPV-associated cancer.

114. The method of claim 113, wherein the HPV-associated cancer is head and neck squamous cell carcinoma.

115. The method of claim 113 or 114, further comprising treating the subject for the HPV-associated cancer.

116. A method for preparing amplified HPV DNA from a biological sample from a subject, the method comprising amplifying HPV DNA from the biological sample using at least six primer pairs comprising at least six of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26.

117. The method of claim 116, wherein the at least six primer pairs comprise at least seven primer pairs comprising at least seven of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs: 19 20, and (xi) SEQ ID NOs:21 and 22, (xii) SEQ ID NOs:23 and 25, and (xiii) SEQ ID NOs:24 and 26.

118. The method of any of claims 116-117117, wherein the at least six primer pairs comprise at least eight primer pairs comprising at least eight of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs: 15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26.

119. The method of any of claims 116-118118, wherein the at least six primer pairs comprise at least nine primer pairs comprising at least nine of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs: 15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26.

120. The method of any of claims 116-119119, wherein the at least six primer pairs comprise at least ten primer pairs comprising at least ten of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26.

121. The method of any of claims 116-120120, wherein the at least six primer pairs comprise at least eleven primer pairs comprising at least eleven of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs: 15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26.

122. The method of any of claims 116-121, wherein the at least six primer pairs comprise at least twelve primer pairs comprising at least twelve of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26.

123. The method of any of claims 116-122, wherein the at least six primer pairs comprise at least thirteen primer pairs comprising (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26.

124. The method of any of claims 116-123, further comprising amplifying genomic DNA from the biological sample using an additional primer pair comprising SEQ ID NOs:27 and 28.

125. The method of any of claims 116-124, wherein the biological sample is a blood, plasma, or serum sample.

126. The method of any of claims 116-125, wherein the HPV DNA is from one or more tumor cells.

127. The method of any of claims 116-125, wherein the HPV DNA is cell-free DNA.

128. The method of any of claims 116-124, wherein the biological sample is a tissue sample.

129. The method of any of claims 116-128, wherein the biological sample contains less than 100 copies of HPV DNA.

130. The method of claim 129, wherein the biological sample contains less than 20 copies of HPV DNA.

131. The method of claim 130, wherein the biological sample contains less than 10 copies of HPV DNA.

132. The method of claim 131, wherein the biological sample contains less than 6 copies of HPV DNA.

133. The method of any of claims 116-132, further comprising, prior to (a), partitioning the HPV DNA into a plurality of partitions.

134. The method of claim 133, wherein the plurality of partitions is a plurality of droplets.

135. A kit for detecting HPV DNA from a subject comprising at least six primer pairs comprising at least six of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26.

136. The kit of claim 135, wherein the at least six primer pairs comprise at least seven primer pairs comprising at least seven of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26.

137. The kit of claim 136, wherein the at least six primer pairs comprise at least eight primer pairs comprising at least eight of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26.

138. The kit of claim 137, wherein the at least six primer pairs comprise at least nine primer pairs comprising at least nine of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26.

139. The kit of claim 138, wherein the at least six primer pairs comprise at least ten primer pairs comprising at least ten of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26.

140. The kit of claim 139, wherein the at least six primer pairs comprise at least eleven primer pairs comprising at least eleven of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26.

141. The kit of claim 140, wherein the at least six primer pairs comprise at least twelve primer pairs comprising at least twelve of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs:19 and 20, (xi) SEQ ID NOs:21 and 22, (xii) SEQ ID NOs:23 and 25, and (xiii) SEQ ID NOs:24 and 26.

142. The kit of claim 141, wherein the at least six primer pairs comprise at least thirteen primer pairs comprising (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs: 13 and 14, (viii) SEQ ID NOs: 15 and 16, (viv) SEQ ID NOs:17 and 18, (x) SEQ ID NOs:19 and 20, (xi) SEQ ID NOs:21 and 22, (xii) SEQ ID NOs:23 and 25, and (xiii) SEQ ID NOs:24 and 26.

143. The kit of any of claims 135-142, further comprising an additional primer pair comprising SEQ ID NOs:27 and 28.

144. The kit of any of claims 135-143, further comprising at least six probes comprising at least six of SEQ ID NOs:29-41.

145. The kit of claim 144, wherein the at least six probes comprise at least seven probes comprising at least seven of SEQ ID NOs:29-41.

146. The kit of claim 145, wherein the at least six probes comprise at least eight probes comprising at least eight of SEQ ID NOs:29-41.

147. The kit of claim 146, wherein the at least six probes comprise at least nine probes comprising at least nine of SEQ ID NOs:29-41.

148. The kit of claim 147, wherein the at least six probes comprise at least ten probes comprising at least ten of SEQ ID NOs:29-41.

149. The kit of claim 148, wherein the at least six probes comprise at least eleven probes comprising at least eleven of SEQ ID NOs:29-41.

150. The kit of claim 149, wherein the at least six probes comprise at least twelve probes comprising at least twelve of SEQ ID NOs:29-41.

151. The kit of claim 150, wherein the at least six probes comprise at least thirteen probes comprising SEQ ID NOs:29-41.

152. The kit of any of claims 135-151, further comprising an additional probe comprising SEQ ID NO:42.

153. A method for detecting HPV DNA in a biological sample from a subject, the method comprising:
(a) amplifying HPV DNA from the biological sample using at least six primer pairs each configured to amplify a different HPV type of a set of high-risk HPV types; and
(b) detecting which HPV types of the set of high-risk HPV types are present in the biological sample;
wherein the set of high-risk HPV types comprises six or more of HPV16, HPV18, HPV31, HPV33, HPV35, HPV39, HPV45, HPV51, HPV52, HPV58, HPV59, HPV56, and HPV68.

154. The method of claim 153, wherein the at least six primer pairs are each configured to amplify a region of the HPV E6 gene or the HPV E7 gene.

155. The method of claim 153, wherein (b) comprises using at least six probes each configured to bind to a different HPV type of the set of high-risk HPV types.

156. The method of claim 153, wherein the set of high-risk HPV types comprises seven or more of HPV16, HPV18, HPV31, HPV33, HPV35, HPV39, HPV45, HPV51, HPV52, HPV58, HPV59, HPV56, and HPV68.

157. The method of claim 156, wherein the set of high-risk HPV types comprises eight or more of HPV16, HPV18, HPV31, HPV33, HPV35, HPV39, HPV45, HPV51, HPV52, HPV58, HPV59, HPV56, and HPV68.

158. The method of claim 157, wherein the set of high-risk HPV types comprises nine or more of HPV16, HPV18, HPV31, HPV33, HPV35, HPV39, HPV45, HPV51, HPV52, HPV58, HPV59, HPV56, and HPV68.

159. The method of claim 158, wherein the set of high-risk HPV types comprises ten or more of HPV16, HPV18, HPV31, HPV33, HPV35, HPV39, HPV45, HPV51, HPV52, HPV58, HPV59, HPV56, and HPV68.

160. The method of claim 159, wherein the set of high-risk HPV types comprises eleven or more of HPV16, HPV18, HPV31, HPV33, HPV35, HPV39, HPV45, HPV51, HPV52, HPV58, HPV59, HPV56, and HPV68.

161. The method of claim 160, wherein the set of high-risk HPV types comprises twelve or more of HPV16, HPV18, HPV31, HPV33, HPV35, HPV39, HPV45, HPV51, HPV52, HPV58, HPV59, HPV56, and HPV68.

162. The method of claim 161, wherein the set of high-risk HPV types comprises HPV16, HPV18, HPV31, HPV33, HPV35, HPV39, HPV45, HPV51, HPV52, HPV58, HPV59, HPV56, and HPV68.

163. The method of claim 153, wherein the at least six primer pairs comprise at least six of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26.

164. The method of claim 163, wherein the at least six primer pairs comprise at least seven primer pairs comprising at least seven of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26.

165. The method of claim 164, wherein the at least six primer pairs comprise at least eight primer pairs comprising at least eight of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs: 13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26.

166. The method of claim 165, wherein the at least six primer pairs comprise at least nine primer pairs comprising at least nine of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26.

167. The method of claim 166, wherein the at least six primer pairs comprise at least ten primer pairs comprising at least ten of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs: 11 and 12, (vii) SEQ ID NOs:13 and 14, (viii) SEQ ID NOs:15 and 16, (viv) SEQ ID NOs: 17 and 18, (x) SEQ ID NOs: 19 and 20, (xi) SEQ ID NOs:21 and 22, (xii) SEQ ID NOs:23 and 25, and (xiii) SEQ ID NOs:24 and 26.

168. The method of claim 167, wherein the at least six primer pairs comprise at least eleven primer pairs comprising at least eleven of (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs:13 and 14, (viii) SEQ ID NOs:15 and 16, (viv) SEQ ID NOs:17 and 18, (x) SEQ ID NOs: 19 and 20, (xi) SEQ ID NOs:21 and 22, (xii) SEQ ID NOs:23 and 25, and (xiii) SEQ ID NOs:24 and 26.

169. The method of claim 168, wherein the at least six primer pairs comprise at least twelve primer pairs comprising at least twelve of (i) SEQ ID NOs:1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs: 13 and 14, (viii) SEQ ID NOs:15 and 16, (viv) SEQ ID NOs: 17 and 18, (x) SEQ ID NOs: 19 and 20, and (xi) SEQ ID NOs:21 and 22, (xii) SEQ ID NOs:23 and 25, and (xiii) SEQ ID NOs:24 and 26.

170. The method of claim 169, wherein the at least six primer pairs comprise at least thirteen primer pairs comprising (i) SEQ ID NOs: 1 and 2, (ii) SEQ ID NOs:3 and 4, (iii) SEQ ID NOs:5 and 6, (iv) SEQ ID NOs:7 and 8, (v) SEQ ID NOs:9 and 10, (vi) SEQ ID NOs:11 and 12, (vii) SEQ ID NOs: 13 and 14, (viii) SEQ ID NOs:15 and 16, (viv) SEQ ID NOs:17 and 18, (x) SEQ ID NOs: 19 and 20, and (xi) SEQ ID NOs:21 and 22, (xii) SEQ ID NOs:23 and 25, and (xiii) SEQ ID NOs:24 and 26.
